(19) United States
(12) Patent Application Publication
(10) Pub. No.: US 2011/0301034 A1
(43) Pub. Date: Dec. 8, 2011

(54) ISOTHIAZOLE AND PYRAZOLE DERIVATIVES FOR USE AS PLANT GROWTH REGULATORS

(75) Inventors: Camilla Corsi, Stein (CH); Sebastian Volker Wendeborn, Stein (CH); Carla Bobbio, Stein (CH); Jilali Kessabi, Stein (CH); Peter Schneiter, Stein (CH); Valeria Grasso, Stein (CH); Ulrich Johannes Haas, Stein (CH)

(73) Assignee: SYNGENTA CROP PROTECTION LLC, Greensboro, NC (US)

(21) Appl. No.: 13/139,042

(22) PCT Filed: Dec. 11, 2009
(86) PCT No.: PCT/EP09/66956

§ 371 Date: Aug. 29, 2011

(30) Foreign Application Priority Data
Dec. 17, 2008 (GB) 0823000.5

(51) Int. Cl. A01N 43/40; C07D 291/04; A01P 21/00; C07D 417/06; A01P 15/00; A01N 43/824; C07D 401/06

(52) U.S. Cl. 504/253; 504/261; 504/252; 548/122; 546/275.4; 546/271.1; 514/359; 514/341; 514/342

(57) ABSTRACT

The present invention relates to isothiazole and pyrazole compounds of formula (I) having plant growth regulating properties, to agricultural compositions comprising them, and to the use of said compounds for regulating plant growth.

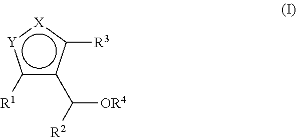

The present invention relates to isothiazole and pyrazole compounds having plant growth regulating properties, to agricultural compositions comprising them, and to the use of said compounds for regulating plant growth.

Plant growth regulators (PGRs) are generally any substances or mixtures of substances intended to accelerate or retard the rate of growth or maturation, or otherwise alter the development of plants or their produce. PGRs affect growth and differentiation of plants, a process which is commonly and hereinafter referred to as “plant health”. There exists a need for further substances having PGR activity.

International patent application WO2009/007098 refers to a series of isothiazole and pyrazole derivatives having fungicidal properties.

It has surprisingly been found that the isothiazole and pyrazole compounds of the present invention exhibit plant growth regulating properties and are therefore suitable for use in agriculture for the improvement and control of plant health.

Accordingly, in a first aspect, the present invention provides for the use of a compound of the formula (I)

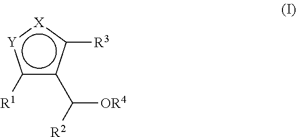

wherein
X is S, N or NR⁵ and Y is N or NR⁵, with the proviso that one, but not both, of X or Y is N;
R¹ and R³ are, independently, hydrogen, or optionally substituted alkyl, alkenyl, alkynyl, heterocyclyl, trialkylsilyl, arylalkyl, aryloxyalkyl, arylthioalkyl, aryl or heteroaryl;
R² is optionally substituted alkyl, alkenyl, alkynyl, heterocyclyl, arylalkyl, aryl or heteroaryl;
R⁴ is H or acyl;
R⁵ is hydrogen or optionally substituted alkyl, alkenyl, alkynyl, heterocyclyl, trialkylsilyl, arylalkyl, aryloxyalkyl, arylthioalkyl, aryl or heteroaryl;
or a salt or N-oxide thereof; as a plant growth regulator.

Plant growth regulators can, for example, reduce plant height, stimulate seed germination, induce flowering, darken leaf coloring, change the rate of plant growth and modify the timing and efficiency of fruiting. In addition, PGRs may exhibit pronounced growth-regulating properties which can result in an increase in the yield of cultivated plants or harvested crops.

PGRs may also have a growth inhibiting action which is dependent on concentration. The growth of both monocots and dicots may be inhibited. Inhibition of the vegetative growth of many cultivated plants permits more plants to be sown in a crop area, so that a higher yield may be obtained per unit of area. Inhibition of the vegetative growth of monocot plants, e.g. cultivated plants such as cereals, is sometimes desirable and advantageous. Such a growth inhibition is of economic interest.

The use of PGRs for inhibiting the growth in height of cereals is also important, as shortening the stalks diminishes or completely eliminates the danger of lodging before harvesting. Additionally, PGRs are able to bring about a strengthening of the stalks in crops of cereals and this too counteracts lodging.

Furthermore, the present invention also provides compositions comprising the isothiazole and pyrazole derivatives of the present invention that improve plants, a process which is commonly and hereinafter referred to as “plant health”.

For example, advantageous properties that may be mentioned are improved crop characteristics including: emergence, crop yield, protein content, increased vigour, faster/delayed maturation, increased speed of seed emergence, improved nutrient utilization efficiency, improved nitrogen utilization efficiency, improved water use efficiency, improved oil content and/or quality, improved digestibility, faster/more even ripening, improved flavor, improved starch content, more developed root system (improved root growth), improved stress tolerance (e.g. against drought, heat, salt, light, UV, water, cold), reduced ethylene (reduced production and/or inhibition of reception), tillering increase, increase in plant height, bigger leaf blade, less dead basal leaves, stronger tillers, greener leaf color, pigment content, photosynthetic activity, less input needed (such as fertilizers or water), less seeds needed, more productive tillers, earlier flowering, early grain maturity, less plant verse (lodging), increased shoot growth, enhanced plant vigor, increased plant stand and early and better germination.

Advantageous properties obtained, especially from treated seeds, include, for example, improved germination and field establishment, better vigor and more homogeneous field establishment.

Advantageous properties obtained, especially from foliar and/or in-furrow application include, for example, improved plant growth and plant development, better growth, more tillers, greener leafes, largers leaves, more biomass, better roots, improved stress tolerance of the plants, more grain yield, more biomass harvested, improved quality of the harvest (content of fatty acids, metabolites, oil etc), more marketable products (e.g. improved size), improved process (e.g. longer shelf-life, better extraction of compounds), improved quality of seeds (for being seeded in the following seasons for seed production); or any other advantages familiar to a person skilled in the art.

It is therefore an object of the present invention to provide compositions and methods suitable for addressing the opportunities outlined above.

The present invention provides plant-protecting active ingredients that are isothiazole and pyrazole compounds of formula (I) according to the invention, in particular the individual isothiazole and pyrazole compounds described in the description as being preferred, and mixtures with increased efficacy and to a method of improving the health of plants by applying said compounds and mixtures to the plants or the locus thereof.

The action of the compounds of formula (I) is separate to any fungicidal action. The isothiazole and pyrazole compounds of formula (I) according to the invention, in particular the individual isothiazole and pyrazole compounds described in the above description as being preferred compounds exhibit plant health properties.

The present invention also concerns compositions comprising or consisting essentially of an active compound as described herein in combination with a suitable carrier (e.g., an agricultural carrier).

The foregoing and other objects and aspects of the present invention are explained in greater detail below.

“Alkyl” as used herein refers to a saturated hydrocarbon radical which may be straight-chain or branched-chain or cyclic (cycloalkyl) and contains from 1 to 24 carbon atoms. This definition applies both when the term is used alone and when it is used as part of a compound term, such as haloalkyl and similar terms. Preferred straight chain and branched alkyl groups may contain 1 to 8 carbon atoms, more preferably 1 to 4 carbons, even more preferably, 1 to 4 carbon atoms. Representative alkyl groups include, for example, methyl, ethyl, isopropyl, n-propyl, n-butyl, t-butyl, t-amyl, and 2,5-dimethylhexyl. Preferred cycloalkyl groups may contain 3 to 12 carbon atoms, more preferably 4 to 10 carbons, even more preferably, 5 to 8 carbon atoms and most preferably 5 or 6 carbon atoms. Preferred cycloalkyl groups include, for example, cyclobutyl, cyclopropyl, cyclopentyl and cyclohexyl.

“Alkenyl” as used herein, refers to a straight or branched chain hydrocarbon containing from 2 to 24 carbons, more preferably 2 to 8 carbons, yet more preferably, 2 to 6 carbon atoms, even more preferably 2 to 4 carbon atoms, and containing at least one carbon-carbon double bond. Representative alkenyl groups include, for example, ethenyl, 2-propenyl, 2-methyl-2-propenyl, 3-butenyl, 4-pentenyl, 5-hexenyl, 2-heptenyl, 2-methyl-1-heptenyl and 3-decenyl.

“Alkynyl” as used herein, refers to a straight or branched chain hydrocarbon group containing from 2 to 24 carbons, more preferably 2 to 8 carbons, yet more preferably, 2 to 6 carbon atoms, even more preferably 2 to 4 carbon atoms, and containing at least one carbon-carbon triple bond. Representative alkynyl groups include, for example, acetylenyl, 1-propynyl, 2-propynyl, 3-butynyl, 2-pentynyl and 1-butynyl.

Representative alkoxy groups include, for example, methoxy, ethoxy and t-butoxy.

Representative alkylthio groups include, for example, methylthio, ethylthio, t-butylthio and hexylthio.

“Aryl” refers to an aromatic substituent which may be a single ring or multiple rings which are fused together, linked covalently or linked to a common group such as an ethylene or methylene moiety. The aromatic rings may each contain heteroatoms and hence aryl encompasses heteroaryl as used herein. Aryl moieties may be optionally substituted with 1 to 4 substituents independently selected from halogen, nitro, alkylcarboxyl, alkoxy and phenoxy. Representative examples of aryl include phenyl azulenyl, indanyl, indenyl, naphthyl, tetrahydronaphthyl, biphenyl, diphenylmethyl, 2,2-diphenyl-1-ethyl, thienyl, pyridyl and quinoxalyl. Most preferably, aryl is phenyl.

“Heteroaryl” means a cyclic, aromatic hydrocarbon containing 3 to 10 ring-atoms including 1 to 4 heteroatoms independently selected from nitrogen, oxygen and sulfur. Preferred heteroaryl groups are five and six membered rings and contain from one to three heteroatoms independently selected from nitrogen, oxygen and sulphur. Heteroaryl moieties may be optionally substituted with 1 to 4 substituents independently selected from halogen, nitro, alkylcarboxyl, alkoxy and phenoxy. Examples of heteroaryl groups include furyl, thienyl, pyrrolyl, oxazolyl, thiazolyl, imidazolyl, pyrazolyl, isoxazolyl, isothiazolyl, oxadiazolyl, triazolyl, thiadiazolyl, pyridyl, pyrimidinyl, pyrazinyl, pyranyl, pyridazinyl, tetrazolyl, triazinyl.

In addition, the term heteroaryl includes fused heteroaryl groups, for example benzimidazolyl, benzoxazolyl, imidazopyridinyl, benzoxazinyl, benzothiazinyl, oxazolopyridinyl, benzofuranyl, quinolinyl, quinazolinyl, quinoxalinyl, benzothiazolyl, phthalimido, benzofuranyl, benzodiazepinyl, indolyl, isoindolyl, isobenzofuranyl, chromenyl, xanthenyl, indolizinyl, indazolyl, purinyl, quinolizinyl, isoquinolyl, phthalazinyl, naphthyridinyl and benzo[b]thienyl.

“Heterocyclyl”, as used herein refers to a saturated or partially unsaturated cyclic hydrocarbon containing from 3 to 10 ring-atoms up to 4 of which may be hetero-atoms such as nitrogen, oxygen and sulfur. Examples of heterocyclyl groups are oxiranyl, azetidinyl, tetrahydrofuranyl, thiolanyl, pyrrolidinyl, pyrrolinyl, imidazolidinyl, imidazolinyl, sulfolanyl, dioxolanyl, dihydropyranyl, tetrahydropyranyl, piperidinyl, pyrazolinyl, pyrazolidinyl, dioxanyl, morpholinyl, dithianyl, thiomorpholinyl, piperazinyl, azepinyl, oxazepinyl, thiazepinyl, thiazolinyl and diazapanyl.

“Acyl” includes any readily hydrolysable acyl groups, and comprises, for example, C(O)R⁷, C(O)OR⁷, C(O)NHR⁷ and C(O)NR⁷R⁸, wherein R⁷ and R⁸ are each independently selected from alkyl, alkenyl, akynyl, heterocyclyl, aryl and heteroaryl. Acyl groups may be optionally substituted with one or more, for example 1, 2, 3 or 4, halo or OR⁷ groups. Preferred acyl groups are acetyl, benzoyl and phenylacetyl.

“Halo” or “halogen” means fluoro, chloro, bromo and iodo and is preferably fluoro or chloro.

“Haloalkyl” includes monohaloalkyl, polyhaloalkyl and perhaloalkyl, for example, chloromethyl, 2-bromoethyl, 2-fluoroethyl, 2,2,2-trifluoroethyl, chlorodifluoromethyl, trichloromethyl, trifluoromethyl, pentafluoroethyl and 2-chloro-3-fluoropentyl.

“Optionally substituted” means substituted by one or more substituents, in particular, one, two, three or four substituents, independently selected from halogen, hydroxyl, cyano, nitro, alkyl, haloalkyl, alkenyl, haloalkenyl, alkynyl, haloalkynyl, heterocyclyl, aryl, heteroaryl, alkoxy, haloalkoxy, alkylthio, haloalkylthio, acyl, alkoxycarbonyl and trialkylsilyl. In the case where groups may be selected from a number of alternative groups, the selected groups may be the same or different.

“Agriculturally acceptable salt” means a salt the cation of which is known and accepted in the art for the formation of salts for agricultural or horticultural use. Preferably the salts are water-soluble.

The compounds of formula (I) may exist in different geometric or optical isomeric forms or in different tautomeric forms. One or more centres of chirality may be present, in which case compounds of the formula (I) may be present as pure enantiomers, mixtures of enantiomers, pure diastereomers or mixtures of diastereomers. There may be double bonds present in the molecule, such as C═C or C═N bonds, in which case compounds of formula (I) may exist as single isomers of mixtures of isomers. Centres of tautomerisation may be present. This invention covers all such isomers and tautomers and mixtures thereof in all proportions as well as isotopic forms such as deuterated compounds.

Suitable salts of the compounds of formula (I), include acid addition salts such as those with an inorganic acid such as hydrochloric, hydrobromic, sulphuric, nitric or phosphoric acid, or an organic carboxylic acid such as oxalic, tartaric, lactic, butyric, toluic, hexanoic or phthalic acid, or a sulphonic acid such as methane, benzene or toluene sulphonic acid. Other examples of organic carboxylic acids include haloacids such as trifluoroacetic acid.

N-oxides are oxidised forms of tertiary amines or oxidised forms of nitrogen containing heteroaromatic compounds. They are described in many books for example in “Heterocyclic N-oxides” by Angelo Albini and Silvio Pietra, CRC Press, Boca Raton, Fla., 1991.

In another aspect, the present invention provides a method of regulating plant growth of crops of useful plants, which comprises applying to said plants, to one or more parts of said plants, or to the locus thereof or plant propagation material, a compound of formula (I) as defined herein.

The preferred embodiments of the invention as defined below apply equally to each aspect and preferred aspects thereof of the invention as defined herein.

In a preferred aspect of the invention, X is S and Y is N, to give the compound of formula (Ia)

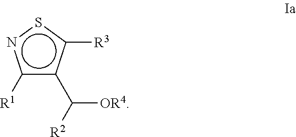

In another preferred aspect of the invention X is NR⁵ and Y is N, to give the compound of formula (Ib)

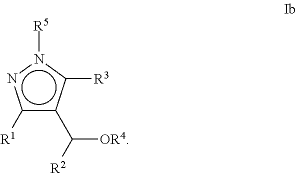

In another preferred aspect of the invention X is N and Y is NR⁵, to give the compound of formula (Ic)

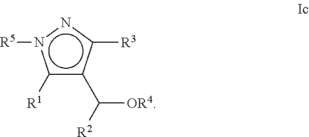

In a preferred embodiment, R¹ is selected from hydrogen; alkyl optionally substituted with phenyl or halophenyl; aryl optionally substituted with halogen, alkyl, haloalkyl, alkoxy, alkylthio, haloalkoxy, cyano or nitro; and heteroaryl optionally substituted with halogen, alkyl, haloalkyl, alkoxy, alkylthio, haloalkoxy, cyano or nitro; or trialkylsilyl.

Preferably, R¹ is selected from hydrogen; C₁-C₆-alkyl optionally substituted with phenyl; phenyl optionally substituted with halogen, C₁-C₆-alkyl, C₁-C₆-haloalkyl, C₁-C₆-alkoxy or C₁-C₆-haloalkoxy; and a 5- or 6-membered heteroaryl optionally substituted with halogen, C₁-C₆-alkyl, C₁-C₆-haloalkyl, C₁-C₆-alkoxy or C₁-C₆-haloalkoxy.

More preferably, R¹ is selected from hydrogen, methyl, ethyl, n-propyl, isopropyl, isoamyl, cyclohexyl, benzyl; phenyl optionally substituted with 1 to 3 groups independently selected from F, Cl, Br, methyl, ethyl, methoxy, ethoxy, trifluoromethyl and trifluoromethoxy; and pyridyl, furyl, thienyl or pyrimidinyl optionally substituted with 1 to 3 groups independently selected from F, Cl, Br, methyl, ethyl, methoxy, ethoxy, trifluoromethyl and trifluoromethoxy.

Yet more preferably, R¹ is selected from n-propyl, isopropyl, cyclohexyl, benzyl; phenyl optionally substituted with 1 or 2 groups independently selected from bromo, chloro, fluoro, methyl, methoxy, trifluoromethyl and trifluoromethoxy; and 2- or 3-pyridyl, 2- or 3-furyl, and 2- or 3-thienyl, each optionally substituted with 1 or 2 chloro.

Even more preferably, R¹ is selected from 2-chlorophenyl, 3-chlorophenyl, 4-chlorophenyl, 4-bromophenyl, 2-fluorophenyl, 3-fluorophenyl, 4-fluorophenyl, 2,4-dichlorophenyl, 2,4-difluorophenyl, 2-fluoro-4-chlorophenyl, 2-chloro-4-fluorophenyl, 2-methylphenyl, 4-methylphenyl, 2,4-dimethylphenyl, 2-methoxyphenyl, 4-methoxyphenyl, 3-trifluoromethylphenyl, 4-trifluoromethylphenyl, 2-chloro-4-methoxyphenyl, 4-methoxytrifluomethylphenyl, 2-methyl-4-chlorophenyl, 2-chloro-3-pyridyl, 2-methoxy-3-pyridyl, 2-thienyl, 3-thienyl and 5-chloro-2-thienyl.

Most preferably, R¹ is 3-chlorophenyl, 4-chlorophenyl, 2-fluorophenyl, 3-fluorophenyl, 4-fluorophenyl, 4-bromophenyl, 2,4-difluorophenyl or 2-thienyl.

In a preferred embodiment, R² is heteroaryl optionally substituted with halogen, alkyl, alkenyl, alkynyl, haloalkyl, alkoxy, alkylthio, haloalkoxy, cyano or nitro.

Preferably, R² is pyridyl or pyrimidinyl, each optionally substituted with halogen, C₁-C₆-alkyl, C₁-C₆-haloalkyl, C₁-C₆-alkoxy or C₁-C₆-haloalkoxy.

More preferably, R² is selected from 2-, 3- or 4-pyridyl and 5-pyrimidinyl, each optionally substituted with halogen, C₁-C₆-haloalkyl or C₁-C₆-alkoxy.

Yet more preferably, R² is selected from 2-pyridyl, 3-pyridyl, and 5-pyrimidinyl, each optionally substituted with methyl, chloro, fluoro or methoxy.

Most preferably, R² is 3-pyridyl or 5-pyrimidinyl.

In another preferred embodiment, R³ is selected from hydrogen; alkyl optionally substituted with phenyl or halophenyl: aryl optionally substituted with halogen, alkyl, haloalkyl, alkoxy, alkylthio, haloalkoxy, cyano or nitro; heteroaryl optionally substituted with halogen, alkyl, haloalkyl, alkoxy, alkylthio, haloalkoxy, cyano or nitro; and trialkylsilyl.

Preferably, R³ is selected from hydrogen; C₁-C₆-alkyl optionally substituted with phenyl; phenyl optionally substituted with halogen, C₁-C₆-alkyl, C₁-C₆-haloalkyl, C₁-C₆-alkoxy or C₁-C₆-haloalkoxy; and a 5- or 6-membered heteroaryl optionally substituted with halogen, C₁-C₆-alkyl, C₁-C₆-haloalkyl, C₁-C₆-alkoxy or C₁-C₆-haloalkoxy.

More preferably, R³ is selected from hydrogen, methyl, ethyl, n-propyl, isopropyl, isoamyl cyclohexyl, benzyl; phenyl optionally substituted with 1 to 3 groups independently selected from bromo, chloro, fluoro, methyl, ethyl, methoxy, ethoxy, trifluoromethyl and trifluoromethoxy; and pyridyl, furyl, thienyl and pyrimidinyl, each optionally substituted with 1 to 3 groups independently selected from bromo, chloro, fluoro, methyl, ethyl, methoxy, ethoxy, trifluoromethyl and trifluoromethoxy.

Yet more preferably, R³ is selected from ethyl, isopropyl, isoamyl, cyclohexyl; phenyl optionally substituted with 1 or 2 groups independently selected from bromo, chloro, fluoro, methoxy and trifluoromethyl; and 2- or 3-furyl, and 2- or 3-thienyl, each optionally substituted with 1 or 2 chloro.

Most preferably, R³ is phenyl, 4-bromophenyl, 3-chlorophenyl, 4-chlorophenyl, 4-fluorophenyl, 2,4-dichlorophenyl, 2,4-difluorophenyl, 2-fluoro-4-chlorophenyl or 4-methoxyphenyl.

In a preferred embodiment, R⁴ is selected from H, acetyl, C(O)Et and C(O)ⁱPr.

Most preferably, R⁴ is H.

In a preferred embodiment R⁵ is selected from hydrogen; alkyl optionally substituted with phenyl or halophenyl: aryl optionally substituted with halogen, alkyl, haloalkyl, alkoxy, alkylthio, haloalkoxy, cyano or nitro; heteroaryl optionally substituted with halogen, alkyl, haloalkyl, alkoxy, alkylthio, haloalkoxy, cyano or nitro; and trialkylsilyl.

More preferably, R⁵ is selected from hydrogen; C₁-C₆-alkyl optionally substituted with phenyl; and phenyl optionally substituted with halogen, C₁-C₆-alkyl, C₁-C₆-haloalkyl, C₁-C₆-alkoxy or C₁-C₆-haloalkoxy.

Yet more preferably, R⁵ is selected from hydrogen, methyl, ethyl, isopropyl, cyclopropyl, benzyl and phenyl.

Most preferably, R⁵ is methyl or benzyl.

In another aspect of the invention, X is S and Y is N

In one preferred aspect of the present invention: X is S and Y is N;

R¹ is selected from hydrogen; C₁-C₆-alkyl optionally substituted with phenyl; phenyl optionally substituted with halogen, C₁-C₆-alkyl, C₁-C₆-haloalkyl, C₁-C₆-alkoxy or C₁-C₆-haloalkoxy; and a 5- or 6-membered heteroaryl optionally substituted with halogen, C₁-C₆-alkyl, C₁-C₆-haloalkyl, C₁-C₆-alkoxy or C₁-C₆-haloalkoxy;
R² is pyridyl or pyrimidinyl, each optionally substituted with halogen, C₁-C₆-alkyl, C₁-C₆-haloalkyl, C₁-C₆-alkoxy or C₁-C₆-haloalkoxy;
R³ is selected from hydrogen; C₁-C₆-alkyl optionally substituted with phenyl; phenyl optionally substituted with halogen, C₁-C₆-alkyl, C₁-C₆-haloalkyl, C₁-C₆-alkoxy or C₁-C₆-haloalkoxy; and a 5- or 6-membered heteroaryl optionally substituted with halogen, C₁-C₆-alkyl, C₁-C₆-haloalkyl, C₁-C₆-alkoxy or C₁-C₆-haloalkoxy; and
R⁴ is selected from H, acetyl, C(O)Et and C(O)ⁱPr.

In a preferred embodiment:

### X is S and Y is N;

R¹ is 3-chlorophenyl, 4-chlorophenyl, 2-fluorophenyl, 2,4-difluorophenyl or 2-thienyl;
R² is 3-pyridyl or 5-pyrimidinyl;
R³ is phenyl, 4-chlorophenyl, 2,4-dichlorophenyl, 4-bromophenyl or 2-fluoro-4-chlorophenyl; and

### R⁴ is H.

In a further aspect of the invention, X is NR⁵ and Y is N.

In another preferred aspect of the present invention: X is NR⁵ and Y is N;

R¹ is selected from hydrogen; C₁-C₆-alkyl optionally substituted with phenyl; phenyl optionally substituted with halogen, C₁-C₆-alkyl, C₁-C₆-haloalkyl, C₁-C₆-alkoxy or C₁-C₆-haloalkoxy; and a 5- or 6-membered heteroaryl optionally substituted with halogen, C₁-C₆-alkyl, C₁-C₆-haloalkyl, C₁-C₆-alkoxy or C₁-C₆-haloalkoxy;
R² is pyridyl or pyrimidinyl, each optionally substituted with halogen, C₁-C₆-alkyl, C₁-C₆-haloalkyl, C₁-C₆-alkoxy or C₁-C₆-haloalkoxy;
R³ is selected from hydrogen; C₁-C₆-alkyl optionally substituted with phenyl; phenyl optionally substituted with halogen, C₁-C₆-alkyl, C₁-C₆-haloalkyl, C₁-C₆-alkoxy or C₁-C₆-haloalkoxy; and a 5- or 6-membered heteroaryl optionally substituted with halogen, C₁-C₆-alkyl, C₁-C₆-haloalkyl, C₁-C₆-alkoxy or C₁-C₆-haloalkoxy;
R⁴ is selected from H, acetyl, C(O)Et and C(O)ⁱPr; and
R⁵ is selected from hydrogen; C₁-C₆-alkyl optionally substituted with phenyl; and phenyl optionally substituted with halogen, C₁-C₆-alkyl, C₁-C₆-haloalkyl, C₁-C₆-alkoxy or C₁-C₆-haloalkoxy.

In a preferred embodiment:

### X is NR⁵ and Y is N;

R¹ is 4-chlorophenyl, 4-bromophenyl, 2-fluorophenyl, 3-fluorophenyl, 4-fluorophenyl or 2,4-difluoro-phenyl;
R² is 3-pyridyl or 5-pyrimidinyl;
R³ is 4-methoxyphenyl, 4-chlorophenyl or 2,4-difluorophenyl;

### R⁴ is H; and

R⁵ is methyl or benzyl.

In a further aspect of the invention, X is N and Y is NR⁵.

In another preferred aspect of the present invention: X is N and Y is NR⁵;

R¹ is selected from hydrogen; C₁-C₆-alkyl optionally substituted with phenyl; phenyl optionally substituted with halogen, C₁-C₆-alkyl, C₁-C₆-haloalkyl, C₁-C₆-alkoxy or C₁-C₆-haloalkoxy; and a 5- or 6-membered heteroaryl optionally substituted with halogen, C₁-C₆-alkyl, C₁-C₆-haloalkyl, C₁-C₆-alkoxy or C₁-C₆-haloalkoxy;
R² is pyridyl or pyrimidinyl, each optionally substituted with halogen, C₁-C₆-alkyl, C₁-C₆-haloalkyl, C₁-C₆-alkoxy or C₁-C₆-haloalkoxy;
R³ is selected from hydrogen; C₁-C₆-alkyl optionally substituted with phenyl; phenyl optionally substituted with halogen, C₁-C₆-alkyl, C₁-C₆-haloalkyl, C₁-C₆-alkoxy or C₁-C₆-haloalkoxy; and a 5- or 6-membered heteroaryl optionally substituted with halogen, C₁-C₆-alkyl, C₁-C₆-haloalkyl, C₁-C₆-alkoxy or C₁-C₆-haloalkoxy;
R⁴ is selected from H, acetyl, C(O)Et and C(O)ⁱPr; and
R⁵ is selected from hydrogen; C₁-C₆-alkyl optionally substituted with phenyl; and phenyl optionally substituted with halogen, C₁-C₆-alkyl, C₁-C₆-haloalkyl, C₁-C₆-alkoxy or C₁-C₆-haloalkoxy.

In a preferred embodiment:

### X is N and Y is NR⁵;

R¹ is 4-chlorophenyl, 2-fluorophenyl, 3-fluorophenyl or 2,4-difluorophenyl;
R² is 3-pyridyl;
R³ is 4-chlorophenyl or 4-fluorophenyl;

### R⁴ is H; and

R⁵ is methyl.

In a further aspect, the present invention provides a method of regulating plant growth of crops of useful plants, which comprises applying to said plants, to one or more parts of said plants, or to the locus thereof or plant propagation material, a compound of formula (I) as defined herein

In a further aspect, the present invention provides a method of regulating plant growth of crops of useful plants, which comprises one or more applications of one of more compounds of formula (I) alone or in conjunction with one or more customary plant protection formulating auxiliaries.

In a further aspect, the present invention provides a method of regulating plant growth of crops of useful plants, which comprises applying to said plants, to one or more parts of said plants, or to the locus thereof or plant propagation material, a compound of formula (I) as defined herein, wherein two or more applications are carried out in sequence, and wherein the two or more applications have the same or different concentration or combinations of compounds as defined herein or both.

In a preferred embodiment of the invention, the crops of useful plants are selected from cereals, rice, beets, leguminous plants, oil plants, cucumber plants, fibre plants, vegetables, plantation crops, ornamentals, vines, bushberries, caneberries, cranberries, peppermint, rhubarb, spearmint, sugar cane and turf grasses.

In a preferred embodiment of the invention, the plant growth regulating effect is an inhibition or a retardation of the plant growth. For example, such a plant growth regulating effect can lead to a plant height decrease.

In another embodiment of the invention, the plant growth regulating effect can lead to a side shoot increase.

In an especially preferred embodiment, the present invention provides an agricultural composition comprising one or more compounds of formula (I) as defined herein and one or more customary plant protection auxiliaries.

In a further aspect, the present invention is directed to the (R)-enantiomers of the compounds of formula (I), designated (R)-(I), wherein R¹, R², R³, R⁴ and R⁵ are as defined herein; and salts thereof.

The present invention provides the compound of formula (R)-(I) as a single enantiomer having an enantiomeric excess (e.e.) of at least 40%, for example, at least 50%, 60%, 70% or 80%, preferably at least 90%, more preferably at least 95%, yet more preferably at least 98% and most preferably at least 99%.

In a further aspect, the present invention is directed to the (R)-enantiomers of the compounds of formula (Ia), designated (R)-(Ia), wherein R¹, R², R³ and R⁴ are as defined herein; and salts thereof.

Preferred compounds of formula (R)-(Ia) include the (R)-enantiomers of compounds A1 to A81 of Table I herein.

The present invention provides the compound of formula (R)-(Ia) as a single enantiomer having an enantiomeric excess (e.e.) of at least 40%, for example, at least 50%, 60%, 70% or 80%, preferably at least 90%, more preferably at least 95%, yet more preferably at least 98% and most preferably at least 99%.

In a further aspect, the present invention is directed to the (S)-enantiomers of the compounds of formula (Ia), designated (S)-(Ia), wherein R¹, R², R³ and R⁴ are as defined herein; and salts thereof.

Preferred compounds of formula (S)-(Ia) include the (S)-enantiomers of compounds A1 to A81 of Table I herein.

The present invention provides the compound of formula (S)-(Ia) as a single enantiomer having an enantiomeric excess (e.e.) of at least 40%, for example, at least 50%, 60%, 70% or 80%, preferably at least 90%, more preferably at least 95%, yet more preferably at least 98% and most preferably at least 99%.

In a further aspect, the present invention is directed to the (R)-enantiomers of the compounds of formula (Ib), designated (R)-(Ib), wherein R¹, R², R³, R⁴ and R⁵ are as defined herein; and salts thereof.

Preferred compounds of formula (R)-(Ib) include the (R)-enantiomers of compounds B1 to B91 of Table II herein.

The present invention provides the compound of formula (R)-(Ib) as a single enantiomer having an enantiomeric excess (e.e.) of at least 40%, for example, at least 50%, 60%, 70% or 80%, preferably at least 90%, more preferably at least 95%, yet more preferably at least 98% and most preferably at least 99%.

In a further aspect, the present invention is directed to the (S)-enantiomers of the compounds of formula (Ib), designated (S)-(Ib), wherein R¹, R², R³, R⁴ and R⁵ are as defined herein; and salts thereof.

Preferred compounds of formula (S)-(Ib) include the (S)-enantiomers of compounds B1 to B81 of Table 1 herein.

The present invention provides the compound of formula (S)-(Ib) as a single enantiomer having an enantiomeric excess (e.e.) of at least 40%, for example, at least 50%, 60%, 70% or 80%, preferably at least 90%, more preferably at least 95%, yet more preferably at least 98% and most preferably at least 99%.

In a further aspect, the present invention is directed to the (R)-enantiomers of the compounds of formula (Ic), designated (R)-(Ic), wherein R¹, R², R³, R⁴ and R⁵ are as defined herein; and salts thereof.

Preferred compounds of formula (R)-(Ic) include the (R)-enantiomers of compounds C1 to C87 of Table III herein.

The present invention provides the compound of formula (R)-(Ic) as a single enantiomer having an enantiomeric excess (e.e.) of at least 40%, for example, at least 50%, 60%, 70% or 80%, preferably at least 90%, more preferably at least 95%, yet more preferably at least 98% and most preferably at least 99%.

In a further aspect, the present invention is directed to the (S)-enantiomers of the compounds of formula (Ic), designated (S)-(Ic), wherein R¹, R², R³, R⁴ and R⁵ are as defined herein; and salts thereof.

Preferred compounds of formula (S)-(Ic) include the (S)-enantiomers of compounds C1 to C87 of Table III herein.

The present invention provides the compound of formula (S)-(Ic) as a single enantiomer having an enantiomeric excess (e.e.) of at least 40%, for example, at least 50%, 60%, 70% or 80%, preferably at least 90%, more preferably at least 95%, yet more preferably at least 98% and most preferably at least 99%.

In another aspect, the present invention relates to an agricultural composition comprising a compound of formula (R)-(I) as defined herein; or an agrochemically acceptable salt thereof, and an agrochemically acceptable diluent or carrier.

In further aspect, the present invention relates to an agricultural composition comprising a compound of formula (S)-(I) as defined herein; or an agrochemically acceptable salt thereof, and an agrochemically acceptable diluent or carrier.

“Plant propagation material” means generative parts of a plant including seeds of all kinds (fruit, tubers, bulbs, grains etc), roots, rhizomes, cuttings, cut shoots and the like. Plant propagation material may also include plants and young plants which are to be transplanted after germination or after emergence from the soil.

“Locus” means the fields on which the plants to be treated are growing, or where the seeds of cultivated plants are sown, or the place where the seed will be placed into the soil.

The “crops of useful plants” to be protected typically comprise, for example, the following species of plants: cereals (wheat, barley, rye, oats, maize (including field corn, pop corn and sweet corn), rice, sorghum and related crops); beet (sugar beet and fodder beet); leguminous plants (beans, lentils, peas, soybeans); oil plants (rape, mustard, sunflowers); cucumber plants (marrows, cucumbers, melons); fibre plants (cotton, flax, hemp, jute); vegetables (spinach, lettuce, asparagus, cabbages, carrots, eggplants, onions, pepper, tomatoes, potatoes, paprika, okra); plantation crops (bananas, fruit trees, rubber trees, tree nurseries), ornamentals (flowers, shrubs, broad-leaved trees and evergreens, such as conifers); as well as other plants such as vines, bushberries (such as blueberries), caneberries, cranberries, peppermint, rhubarb, spearmint, sugar cane and turf grasses including, for example, cool-season turf grasses (for example, bluegrasses (Poa L.), such as Kentucky bluegrass (Poa pratensis L.), rough bluegrass (Poa trivialis L.), Canada bluegrass (Poa compressa L.) and annual bluegrass (Poa annua L.); bentgrasses (Agrostis L.), such as creeping bentgrass (Agrostis palustris Huds.), colonial bentgrass (Agrostis tenius Sibth.), velvet bentgrass (Agrostis canina L.) and redtop (Agrostis alba L.); fescues (Festuca L.), such as tall fescue (Festuca arundinacea Schreb.), meadow fescue (Festuca elatior L.) and fine fescues such as creeping red fescue (Festuca rubra L.), chewings fescue (Festuca rubra var. commutate Gaud.), sheep fescue (Festuca ovina L.) and hard fescue (Festuca longifolia); and ryegrasses (Lolium L.), such as perennial ryegrass (Lolium perenne L.) and annual (Italian) ryegrass (Lolium multiflorum Lam.)) and warm-season turf grasses (for example, Bermudagrasses (Cynodon L. C. Rich), including hybrid and common Bermudagrass; Zoysiagrasses (Zoysia Willd.), St. Augustinegrass (Stenotaphrum secundatum (Walt.) Kuntze); and centipedegrass (Eremochloa ophiuroides (Munro.) Hack.)).

The term “useful plants” also includes also useful plants that have been rendered tolerant to herbicides like bromoxynil or classes of herbicides (such as, for example, HPPD inhibitors, ALS inhibitors, for example primisulfuron, prosulfuron and trifloxysulfuron, EPSPS (5-enol-pyrovyl-shikimate-3-phosphate-synthase) inhibitors, GS (glutamine synthetase) inhibitors or PPO (protoporphyrinogen-oxidase) inhibitors) as a result of conventional methods of breeding or genetic engineering. An example of a crop that has been rendered tolerant to imidazolinones, e.g. imazamox, by conventional methods of breeding (mutagenesis) is Clearfield® summer rape (Canola). Examples of crops that have been rendered tolerant to herbicides or classes of herbicides by genetic engineering methods include glyphosate- and glufosinate-resistant maize varieties commercially available under the trade names RoundupReady®, Herculex I®® and LibertyLink®.

The term “useful plants” also includes useful plants which have been so transformed by the use of recombinant DNA techniques that they are capable of synthesising one or more selectively acting toxins, such as are known, for example, from toxin-producing bacteria, especially those of the genus Bacillus.

The term “useful plants” also includes useful plants which have been so transformed by the use of recombinant DNA techniques that they are capable of synthesising antipathogenic substances having a selective action, such as, for example, the so-called “pathogenesis-related proteins” (PRPs, see e.g. EP-A-0 392 225). Examples of such antipathogenic substances and transgenic plants capable of synthesising such antipathogenic substances are known, for example, from EP-A-0 392 225, WO 95/33818, and EP-A-0 353 191. The methods of producing such transgenic plants are generally known to the person skilled in the art and are described, for example, in the publications mentioned above.

The agrochemical composition of the present invention will usually contain from 0.1 to 99% by weight, preferably from 0.1 to 95% by weight, of the compound of formula (I), 99.9 to 1% by weight, preferably 99.8 to 5% by weight, of a solid or liquid adjuvant, and from 0 to 25% by weight, preferably from 0.1 to 25% by weight, of a surfactant.

Suitably, the agrochemical compositions of the present invention are applied prior to disease development. Rates and frequency of use of the formulations are those conventionally used in the art and will depend on factors such as the developmental stage of the plant and on the location, timing and application method. Advantageous rates of application are normally from 5 g to 2 kg of active ingredient (a.i.) per hectare (ha), preferably from 10 g to 1 kg a.i./ha, most preferably from 20 g to 600 g a.i./ha. When used as seed drenching agent, convenient rates of application are from 10 mg to 1 g of active substance per kg of seeds.

In practice, as indicated above, the agrochemical compositions comprising compound of formula (I) are applied as a formulation containing the various adjuvants and carriers known to or used in the industry. They may thus be formulated as granules, as wettable or soluble powders, as emulsifiable concentrates, as coatable pastes, as dusts, as flowables, as solutions, as suspensions or emulsions, or as controlled release forms such as microcapsules. These formulations are described in more detail below and may contain as little as about 0.5% to as much as about 95% or more by weight of the active ingredient. The optimum amount will depend on formulation, application equipment and nature of the plant to be treated.

Suspension concentrates are aqueous formulations in which finely divided solid particles of the active compound are suspended. Such formulations include anti-settling agents and dispersing agents and may further include a wetting agent to enhance activity as well an anti-foam and a crystal growth inhibitor. In use, these concentrates are diluted in water and normally applied as a spray to the area to be treated. The amount of active ingredient may range from about 0.5% to about 95% of the concentrate.

Wettable powders are in the form of finely divided particles which disperse readily in water or other liquid carriers. The particles contain the active ingredient retained in a solid matrix. Typical solid matrices include fuller's earth, kaolin clays, silicas and other readily wet organic or inorganic solids. Wettable powders normally contain about 5% to about 95% of the active ingredient plus a small amount of wetting, dispersing or emulsifying agent.

Emulsifiable concentrates are homogeneous liquid compositions dispersible in water or other liquid and may consist entirely of the active compound with a liquid or solid emulsifying agent, or may also contain a liquid carrier, such as xylene, heavy aromatic naphthas, isophorone and other non-volatile organic solvents. In use, these concentrates are dispersed in water or other liquid and normally applied as a spray to the area to be treated. The amount of active ingredient may range from about 0.5% to about 95% of the concentrate.

Granular formulations include both extrudates and relatively coarse particles and are usually applied without dilution to the area in which treatment is required. Typical carriers for granular formulations include sand, fuller's earth, attapulgite clay, bentonite clays, montmorillonite clay, vermiculite, perlite, calcium carbonate, brick, pumice, pyrophyllite, kaolin, dolomite, plaster, wood flour, ground corn cobs, ground peanut hulls, sugars, sodium chloride, sodium sulphate, sodium silicate, sodium borate, magnesia, mica, iron oxide, zinc oxide, titanium oxide, antimony oxide, cryolite, gypsum, diatomaceous earth, calcium sulphate and other organic or inorganic materials which absorb or which can be coated with the active compound. Granular formulations normally contain about 5% to about 25% active ingredients which may include surface-active agents such as heavy aromatic naphthas, kerosene and other petroleum fractions, or vegetable oils; and/or stickers such as dextrins, glue or synthetic resins.

Dusts are free-flowing admixtures of the active ingredient with finely divided solids such as talc, clays, flours and other organic and inorganic solids which act as dispersants and carriers.

Microcapsules are typically droplets or granules of the active ingredient enclosed in an inert porous shell which allows escape of the enclosed material to the surroundings at controlled rates. Encapsulated droplets are typically about 1 to 50 microns in diameter. The enclosed liquid typically constitutes about 50 to 95% of the weight of the capsule and may include solvent in addition to the active compound. Encapsulated granules are generally porous granules with porous membranes sealing the granule pore openings, retaining the active species in liquid form inside the granule pores. Granules typically range from 1 millimetre to 1 centimetre and preferably 1 to 2 millimetres in diameter. Granules are formed by extrusion, agglomeration or prilling, or are naturally occurring. Examples of such materials are vermiculite, sintered clay, kaolin, attapulgite clay, sawdust and granular carbon. Shell or membrane materials include natural and synthetic rubbers, cellulosic materials, styrene-butadiene copolymers, polyacrylonitriles, polyacrylates, polyesters, polyamides, polyureas, polyurethanes and starch xanthates.

Other useful formulations for agrochemical applications include simple solutions of the active ingredient in a solvent in which it is completely soluble at the desired concentration, such as acetone, alkylated naphthalenes, xylene and other organic solvents. Pressurised sprayers, wherein the active ingredient is dispersed in finely-divided form as a result of vaporisation of a low boiling dispersant solvent carrier, may also be used.

Suitable agricultural adjuvants and carriers that are useful in formulating the compositions of the invention in the formulation types described above are well known to those skilled in the art. Suitable examples of the different classes are found below.

Liquid carriers that can be employed include water, toluene, xylene, petroleum naphtha, crop oil, acetone, methyl ethyl ketone, cyclohexanone, acetic anhydride, acetonitrile, acetophenone, amyl acetate, 2-butanone, chlorobenzene, cyclohexane, cyclohexanol, alkyl acetates, diacetonalcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethyl formamide, dimethyl sulfoxide, 1,4-dioxane, dipropylene glycol, dipropylene glycol methyl ether, dipropylene glycol dibenzoate, diproxitol, alkyl pyrrolidinone, ethyl acetate, 2-ethyl hexanol, ethylene carbonate, 1,1,1-trichloroethane, 2-heptanone, alpha pinene, d-limonene, ethylene glycol, ethylene glycol butyl ether, ethylene glycol methyl ether, gamma-butyrolactone, glycerol, glycerol diacetate, glycerol monoacetate, glycerol triacetate, hexadecane, hexylene glycol, isoamyl acetate, isobornyl acetate, isooctane, isophorone, isopropyl benzene, isopropyl myristate, lactic acid, laurylamine, mesityl oxide, methoxy-propanol, methyl isoamyl ketone, methyl isobutyl ketone, methyl laurate, methyl octanoate, methyl oleate, methylene chloride, m-xylene, n-hexane, n-octylamine, octadecanoic acid, octyl amine acetate, oleic acid, oleylamine, o-xylene, phenol, polyethylene glycol (PEG400), propionic acid, propylene glycol, propylene glycol monomethyl ether, p-xylene, toluene, triethyl phosphate, triethylene glycol, xylene sulfonic acid, paraffin, mineral oil, trichloroethylene, perchloroethylene, ethyl acetate, amyl acetate, butyl acetate, methanol, ethanol, isopropanol, and higher molecular weight alcohols such as amyl alcohol, tetrahydrofurfuryl alcohol, hexanol, octanol, etc. ethylene glycol, propylene glycol, glycerine, N-methyl-2-pyrrolidinone, and the like. Water is generally the carrier of choice for the dilution of concentrates.

Suitable solid carriers include talc, titanium dioxide, pyrophyllite clay, silica, attapulgite clay, kieselguhr, chalk, diatomaxeous earth, lime, calcium carbonate, bentonite clay, fuller's earth, cotton seed hulls, wheat flour, soybean flour, pumice, wood flour, walnut shell flour, lignin and the like.

A broad range of surface-active agents are advantageously employed in both said liquid and solid compositions, especially those designed to be diluted with carrier before application. These agents, when used, normally comprise from 0.1% to 15% by weight of the formulation. They can be anionic, cationic, non-ionic or polymeric in character and can be employed as emulsifying agents, wetting agents, suspending agents or for other purposes. Typical surface active agents include salts of alkyl sulfates, such as diethanolammonium lauryl sulphate; alkylarylsulfonate salts, such as calcium dodecylbenzenesulfonate; alkylphenol-alkylene oxide addition products, such as nonylphenol-C.sub. 18 ethoxylate; alcohol-alkylene oxide addition products, such as tridecyl alcohol-C.sub. 16 ethoxylate; soaps, such as sodium stearate; alkylnaphthalenesulfonate salts, such as sodium dibutylnaphthalenesulfonate; dialkyl esters of sulfosuccinate salts, such as sodium di(2-ethylhexyl)sulfosuccinate; sorbitol esters, such as sorbitol oleate; quaternary amines, such as lauryl trimethylammonium chloride; polyethylene glycol esters of fatty acids, such as polyethylene glycol stearate; block copolymers of ethylene oxide and propylene oxide; and salts of mono and dialkyl phosphate esters.

Other adjuvants commonly utilized in agricultural compositions include crystallisation inhibitors, viscosity modifiers, suspending agents, spray droplet modifiers, pigments, antioxidants, foaming agents, anti-foaming agents, light-blocking agents, compatibilizing agents, antifoam agents, sequestering agents, neutralising agents and buffers, corrosion inhibitors, dyes, odorants, spreading agents, penetration aids, micronutrients, emollients, lubricants, sticking agents, and the like.

In addition, further, other biocidally active ingredients or compositions may be combined with the compound of formula (I) and used in the methods of the invention and applied simultaneously or sequentially with the compound of formula (I). When applied simultaneously, these further active ingredients may be formulated together with the compound of formula (I) or mixed in, for example, the spray tank. These further biocidally active ingredients may be fungicides, herbicides, insecticides, bactericides, acaricides, nematicides and/or plant growth regulators.

Accordingly, the present invention provides for the use of a composition in the methods of the present invention, said composition comprising a compound of formula (I) as defined herein, and (i) a fungicide, (ii) a herbicide, (iii) an insecticide, (iv) a bactericide, (v) an acaricide, (vi) a nematicide and/or (vii) a plant growth regulator.

In one aspect, the present invention provides for the use of a composition in the methods of the present invention, said composition comprising a compound of formula (Ia) as defined herein, or compounds A1 to A81 of Table I, and (i) a fungicide, (ii) a herbicide, (iii) an insecticide, (iv) a bactericide, (v) an acaricide, (vi) a nematicide and/or (vii) a plant growth regulator.

In one embodiment, the present invention provides for the use of a composition in the methods of the present invention, said composition comprising a compound of formula (Ia) which is the (R)-enantiomer of formula (R)-(Ia), or the (R)-enantiomer of compounds A1 to A81, of Table I and (i) a fungicide, (ii) a herbicide, (iii) an insecticide, (iv) a bactericide, (v) an acaricide, (vi) a nematicide and/or (vii) a plant growth regulator.

In a further embodiment, the present invention provides for the use of a composition in the methods of the present invention, said composition comprising a compound of formula (Ia) which is the (S)-enantiomer of formula (S)-(Ia), or the (S)-enantiomer of compounds A1 to A81, of Table I and (i) a fungicide, (ii) a herbicide, (iii) an insecticide, (iv) a bactericide, (v) an acaricide, (vi) a nematicide and/or (vii) a plant growth regulator.

In one aspect, the present invention provides for the use of a composition in the methods of the present invention, said composition comprising a compound of formula (Ib) as defined herein, or compounds B1 to B91 of Table II, and (i) a fungicide, (ii) a herbicide, (iii) an insecticide, (iv) a bactericide, (v) an acaricide, (vi) a nematicide and/or (vii) a plant growth regulator.

In one embodiment, the present invention provides for the use of a composition in the methods of the present invention, said composition comprising a compound of formula (Ib) which is the (R)-enantiomer of formula (R)-(Ib), or the (R)-enantiomer of compounds B1 to B91 of Table II, and (i) a fungicide, (ii) a herbicide, (iii) an insecticide, (iv) a bactericide, (v) an acaricide, (vi) a nematicide and/or (vii) a plant growth regulator.

In a further embodiment, the present invention provides for the use of a composition in the methods of the present invention, said composition comprising a compound of formula (Ib) which is the (S)-enantiomer of formula (S)-(Ib), or the (S)-enantiomer of compounds B1 to B91, of Table II and (i) a fungicide, (ii) a herbicide, (iii) an insecticide, (iv) a bactericide, (v) an acaricide, (vi) a nematicide and/or (vii) a plant growth regulator.

In one aspect, the present invention provides for the use of a composition in the methods of the present invention, said composition comprising a compound of formula (Ic) as defined herein, or compounds C1 to C87 of Table III, and (i) a fungicide, (ii) a herbicide, (iii) an insecticide, (iv) a bactericide, (v) an acaricide, (vi) a nematicide and/or (vii) a plant growth regulator.

In one embodiment, the present invention provides for the use of a composition in the methods of the present invention, said composition comprising a compound of formula (Ic) which is the (R)-enantiomer of formula (R)-(Ic), or the (R)-enantiomer of compounds C1 to C87 of Table III, and (i) a fungicide, (ii) a herbicide, (iii) an insecticide, (iv) a bactericide, (v) an acaricide, (vi) a nematicide and/or (vii) a plant growth regulator.

In a further embodiment, the present invention provides for the use of a composition in the methods of the present invention, said composition comprising a compound of formula (Ic) which is the (S)-enantiomer of formula (S)-(Ic), or the (S)-enantiomer of compounds C1 to C87 of Table III, and (i) a fungicide, (ii) a herbicide, (iii) an insecticide, (iv) a bactericide, (v) an acaricide, (vi) a nematicide and/or (vii) a plant growth regulator.

In addition, the compounds of the invention may also be applied with one or more systemically acquired resistance inducers (“SAR” inducer). SAR inducers are known and described in, for example, U.S. Pat. No. 6,919,298 and include, for example, salicylates and the commercial SAR inducer acibenzolar-5-methyl.

In particular, composition encompassed by the present invention include, for example, compositions comprising a compound of formula (I) and acibenzolar (CGA245704), a compound of formula (I) and ancymidol, a compound of formula (I) and alanycarb, a compound of formula (I) and aldimorph, a compound of formula (I) and amisulbrom, a compound of formula (I) and anilazine, a compound of formula (I) and azaconazole, a compound of formula (I) and azoxystrobin, a compound of formula (I) and benalaxyl, a compound of formula (I) and benthiavalicarb, a compound of formula (I) and benomyl, a compound of formula (I) and biloxazol, a compound of formula (I) and bitertanol, a compound of formula (I) and bixafen, a compound of formula (I) and blasticidin S, a compound of formula (I) and boscalid, a compound of formula (I) and bromuconazole, a compound of formula (I) and bupirimate, a compound of formula (I) and captafol, a compound of formula (I) and captan, a compound of formula (I) and carbendazim, a compound of formula (I) and carbendazim, a compound of formula (I) and chlorhydrate, a compound of formula (I) and carboxin, a compound of formula (I) and carpropamid, a compound of formula (I) and carvone, a compound of formula (I) and CGA41396, a compound of formula (I) and CGA41397, a compound of formula (I) and chinomethionate, a compound of formula (I) and chloroneb, a compound of formula (I) and chlorothalonil, a compound of formula (I) and chlorozolinate, a compound of formula (I) and clozylacon, a compound of formula (I) and copper containing compounds such as copper oxychloride, copper oxyquinolate, copper sulphate, copper tallate and Bordeaux mixture, a compound of formula (I) and cyflufenamid, a compound of formula (I) and cymoxanil, a compound of formula (I) and cyproconazole, a compound of formula (I) and cyprodinil, a compound of formula (I) and debacarb, a compound of formula (I) and di-2-pyridyl disulphide 1,1′-dioxide, a compound of formula (I) and dichlofluanid, a compound of formula (I) and diclomezine, a compound of formula (I) and dichlozoline, a compound of formula (I) and dichlone, a compound of formula (I) and dicloran, a compound of formula (I) and diclocymet, a compound of formula (I) and diethofencarb, a compound of formula (I) and difenoconazole, a compound of formula (I) and difenzoquat, a compound of formula (I) and diflumetorim, a compound of formula (I) and O,O-di-iso-propyl-S-benzyl thiophosphate, a compound of formula (I) and dimefluazole, a compound of formula (I) and dimetconazole, a compound of formula (I) and dimethomorph, a compound of formula (I) and dimethirimol, a compound of formula (I) and dimoxystrobin, a compound of formula (I) and diniconazole, a compound of formula (I) and dinocap, a compound of formula (I) and dithianon, a compound of formula (I) and dodecyl dimethyl ammonium chloride, a compound of formula (I) and dodemorph, a compound of formula (I) and dodine, a compound of formula (I) and doguadine, a compound of formula (I) and edifenphos, a compound of formula (I) and enestrobin, a compound of formula (I) and epoxiconazole, a compound of formula (I) and ethaboxam, a compound of formula (I) and ethirimol, a compound of formula (I) and etridiazole, a compound of formula (I) and famoxadone, a compound of formula (I) and fenamidone (RPA407213), a compound of formula (I) and fenarimol, a compound of formula (I) and fenbuconazole, a compound of formula (I) and fenfuram, a compound of formula (I) and fenhexamid (KBR2738), a compound of formula (I) and fenoxanil, a compound of formula (I) and fenpiclonil, a compound of formula (I) and fenpropidin, a compound of formula (I) and fenpropimorph, a compound of formula (I) and fentin acetate, a compound of formula (I) and fentin hydroxide, a compound of formula (I) and ferbam, a compound of formula (I) and ferimzone, a compound of formula (I) and fluazinam, a compound of formula (I) and fluopicolide, a compound of formula (I) and fludioxonil, a compound of formula (I) and fluoxastrobin, a compound of formula (I) and flumetover, a compound of formula (I) and SYP-L190 (flumorph), a compound of formula (I) and fluopyram, a compound of formula (I) and fluoroimide, a compound of formula (I) and fluquinconazole, a compound of formula (I) and flusilazole, a compound of formula (I) and flusulfamide, a compound of formula (I) and flutolanil, a compound of formula (I) and flutriafol, a compound of formula (I) and folpet, a compound of formula (I) and fosetyl-aluminium, a compound of formula (I) and fuberidazole, a compound of formula (I) and furalaxyl, a compound of formula (I) and furametpyr, a compound of formula (I) and guazatine, a compound of formula (I) and hexaconazole, a compound of formula (I) and hydroxyisoxazole, a compound of formula (I) and hymexazole, a compound of formula (I) and IKF-916 (cyazofamid), a compound of formula (I) and imazalil, a compound of formula (I) and imibenconazole, a compound of formula (I) and iminoctadine, a compound of formula (I) and iminoctadine triacetate, a compound of formula (I) and ipconazole, a compound of formula (I) and iprobenfos, a compound of formula (I) and iprodione, a compound of formula (I) and iprovalicarb (SZX0722), a compound of formula (I) and isopropanyl butyl carbamate, a compound of formula (I) and isoprothiolane, a compound of formula (I) and kasugamycin, a compound of formula (I) and kresoxim-methyl, a compound of formula (I) and LY186054, a compound of formula (I) and LY211795, a compound of formula (I) and LY248908, a compound of formula (I) and maneb, a compound of formula (I) and mancopper, a compound of formula (I) and man-cozeb, a compound of formula (I) and mandipropamid, a compound of formula (I) and mefenoxam, a compound of formula (I) and mepanipyrim, a compound of formula (I) and mepronil, a compound of formula (I) and metalaxyl, a compound of formula (I) and metconazole, a compound of formula (I) and methasulfocarb, a compound of formula (I) and metiram, a compound of formula (I) and metiram-zinc, a compound of formula (I) and metominostrobin, a compound of formula (I) and metrafenone, a compound of formula (I) and myclobutanil, a compound of formula (I) and myclozoline, a compound of formula (I) and neoasozin, a compound of formula (I) and nickel dimethyldithiocarbamate, a compound of formula (I) and nitrothal-isopropyl, a compound of formula (I) and nuarimol, a compound of formula (I) and ofurace, a compound of formula (I) and organomercury compounds, a compound of formula (I) and orysastrobin, a compound of formula (I) and oxadixyl, a compound of formula (I) and oxasulfuron, a compound of formula (I) and oxine-copper, a compound of formula (I) and oxolinic acid, a compound of formula (I) and oxpoconazole, a compound of formula (I) and oxycarboxin, a compound of formula (I) and pefurazoate, a compound of formula (I) and penconazole, a compound of formula (I) and pencycuron, a compound of formula (I) and penthiopyrad, a compound of formula (I) and phenazin oxide, a compound of formula (I) and phosdiphen, a compound of formula (I) and phosphorus acids, a compound of formula (I) and phthalide, a compound of formula (I) and picoxystrobin (ZA1963), a compound of formula (I) and polyoxin D, a compound of formula (I) and polyram, a compound of formula (I) and probenazole, a compound of formula (I) and prochloraz, a compound of formula (I) and procymidone, a compound of formula (I) and propamocarb, a compound of formula (I) and propiconazole, a compound of formula (I) and propineb, a compound of formula (I) and propionic acid, a compound of formula (I) and proquinazid, a compound of formula (I) and prothioconazole, a compound of formula (I) and pyraclostrobin, a compound of formula (I) and pyrazophos, a compound of formula (I) and pyribencarb, a compound of formula (I) and pyrifenox, a compound of formula (I) and pyrimethanil, a compound of formula (I) and pyroquilon, a compound of formula (I) and pyroxyfur, a compound of formula (I) and pyrrolnitrin, a compound of formula (I) and quaternary ammonium compounds, a compound of formula (I) and quinomethionate, a compound of formula (I) and quinoxyfen, a compound of formula (I) and quintozene, a compound of formula (I) and silthiofam, a compound of formula (I) and simeconazole, a compound of formula (I) and sipconazole (F-155), a compound of formula (I) and sodium pentachlorophenate, a compound of formula (I) and spiroxamine, a compound of formula (I) and streptomycin, a compound of formula (I) and sulphur, a compound of formula (I) and tebuconazole, a compound of formula (I) and tecloftalam, a compound of formula (I) and tecnazene, a compound of formula (I) and tetraconazole, a compound of formula (I) and thiabendazole, a compound of formula (I) and thifluzamid, a compound of formula (I) and 2-(thiocyanomethylthio)benzothiazole, a compound of formula (I) and thiophanate-methyl, a compound of formula (I) and thiram, a compound of formula (I) and tiadinil, a compound of formula (I) and timibenconazole, a compound of formula (I) and tolclofos-methyl, a compound of formula (I) and tolylfluanid, a compound of formula (I) and triadimefon, a compound of formula (I) and triadimenol, a compound of formula (I) and triazbutil, a compound of formula (I) and triazoxide, a compound of formula (I) and tricyclazole, a compound of formula (I) and tridemorph, a compound of formula (I) and trifloxystrobin (CGA279202), a compound of formula (I) and triforine, a compound of formula (I) and triflumizole, a compound of formula (I) and triticonazole, a compound of formula (I) and validamycin A, a compound of formula (I) and vapam, a compound of formula (I) and valiphenal a compound of formula (I) and vinclozolin, a compound of formula (I) and zineb, a compound of formula (I) and ziram, a compound of formula (I) and zoxamide, a compound of formula (I) and 3-[5-(4-chlorophenyl)-2,3-dimethylisoxazolidin-3-yl]pyridine, a compound of formula (I) and 5-chloro-7-(4-methylpiperidine-1-yl)-6-(2,4,6-trifluorophenyl)[1,2,4]triazolo[1,5-a]pyrimidine and a compound of formula (I) and N-(4-chloro-2-nitrophenyl)-N-ethyl-4-methyl-benzsulfonamide.

Plant growth regulators (PGRs) affect growth and differentiation of plants.

More specifically, various plant growth regulators (PGRs) can, for example, reduce plant height, stimulate seed germination, induce flowering, darken leaf coloring, change the rate of plant growth and modify the timing and efficiency of fruiting.

In addition plant growth regulators (PGRs) may exhibit pronounced growth-regulating properties which can result in an increase in the yield of cultivated plants or harvested crops.

Further, plant growth regulators (PGRs) may have a growth inhibiting action which is dependent on the concentration. The growth of both monocots and dicots may be inhibited. Inhibition of the vegetative growth of many cultivated plants permits more plants to be sown in a crop area, so that a higher yield may be obtained per unit of area. Inhibition of the vegetative growth of monocot plants, e.g. cultivated plants such as cereals, is sometimes desirable and advantageous. Such a growth inhibition is of economic interest.

The use of plant growth regulators (PGRs) for inhibiting the growth in height of cereals is also important, as shortening the stalks diminishes or completely eliminates the danger of lodging before harvesting.

In addition, plant growth regulators (PGRs) are able to bring about a strengthening of the stalks in crops of cereals and this too counteracts lodging.

Furthermore, the present invention also relates to compositions comprising the isothiazole and pyrazole derivatives of the present invention that improve plants, a process which is commonly and hereinafter referred to as “plant health”.

For example, advantageous properties that may be mentioned are improved crop characteristics including: emergence, crop yield, protein content, increased vigour, faster/delayed maturation, increased speed of seed emergence, improved nutrient utilization efficiency, improved nitrogen utilization efficiency, improved water use efficiency, improved oil content and/or quality, improved digestibility, faster/more even ripening, improved flavor, improved starch content, more developed root system (improved root growth), improved stress tolerance (e.g. against drought, heat, salt, light, UV, water, cold), reduced ethylene (reduced production and/or inhibition of reception), tillering increase, increase in plant height, bigger leaf blade, less dead basal leaves, stronger tillers, greener leaf color, pigment content, photosynthetic activity, less input needed (such as fertilizers or water), less seeds needed, more productive tillers, earlier flowering, early grain maturity, less plant verse (lodging), increased shoot growth, enhanced plant vigor, increased plant stand and early and better germination.

Advantageous properties, obtained especially from treated seeds, are e.g. improved germination and field establishment, better vigor, more homogeneous field establishment.

Advantageous properties, obtained especially from foliar and/or in-furrow application are e.g. improved plant growth and plant development, better growth, more tillers, greener leafes, largers leaves, more biomass, better roots, improved stress tolerance of the plants, more grain yield, more biomass harvested, improved quality of the harvest (content of fatty acids, metabolites, oil etc), more marketable products (e.g. improved size), improved process (e.g. longer shelf-life, better extraction of compounds), improved quality of seeds (for being seeded in the following seasons for seed production); or any other advantages familiar to a person skilled in the art.

It is therefore an object of the present invention to provide a method which solves the problems outlined above.

The present invention relates to plant-protecting active ingredients that are isothiazole and pyrazole compounds of formula (I) according to the invention, in particular the individual isothiazole and pyrazole compounds described in the above description as being preferred, and mixtures with increased efficacy and to a method of improving the health of plants by applying said compounds and mixtures to the plants or the locus thereof.

The action of the compounds of formula (I) goes beyond the known fungicidal action. The isothiazole and pyrazole compounds of formula (I) according to the invention, in particular the individual isothiazole and pyrazole compounds described in the above description as being preferred compounds exhibit plant health.

### Preparation of Compounds of Formula (I)a

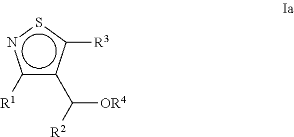

Methods for the preparation of isothiazoles are reviewed in Science of Synthesis (2002), 11, 507-572.

3-Substituted isothiazoles can be prepared by 1,3-dipolar cycloaddition, as described in Synthetic Communications, 35(6), 807, 2005 or ARKIVOC (3), 121, 2002.

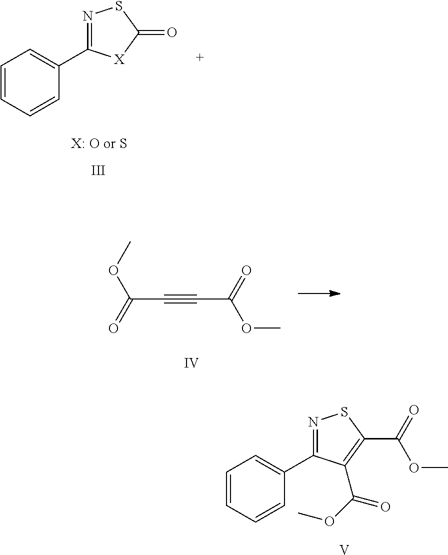

Compounds of type V are converted to final products as described in the Examples.

Other useful intermediates for the preparation of compounds of type Ia are found in the following journals:
Chemistry Letters; 1984, 1691-92:

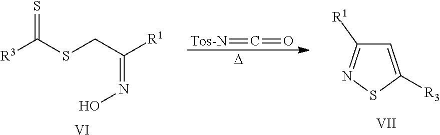

Journal of Heterocyclic Chemistry; 1989, 1575:

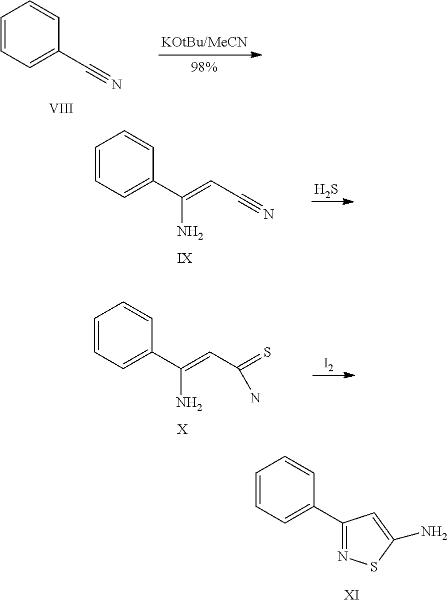

Pd-catalyzed cross coupling reactions can be used to convert 3,5-dihalo-isothiazole-4-carbonitriles XII into final compounds of formula (I)a. Experimental details for such transformations can be found in Perk I, 2006, 3681:

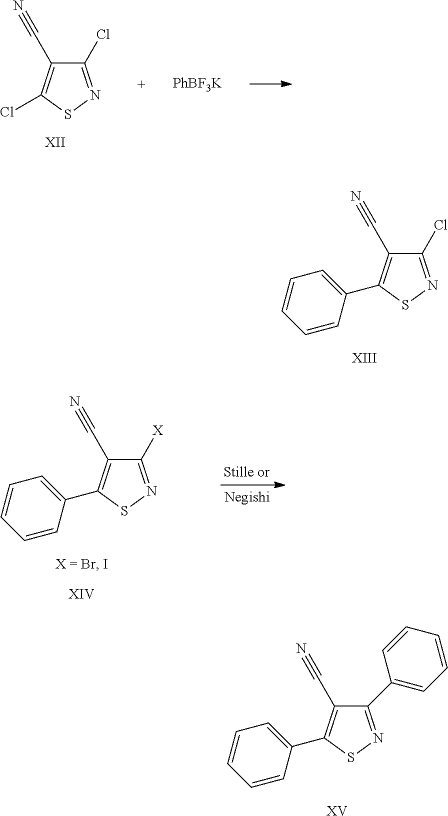

Preparation of compounds of formula (I)b and Ic

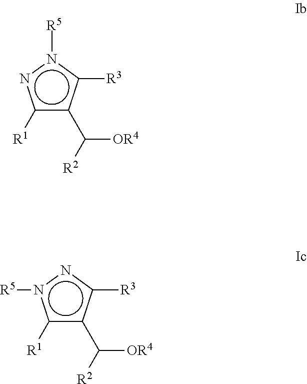

N-Substituted pyrazoles XVII can easily be prepared usually as a mixture of two isomers by the reaction of 1,3-diketones (XVI) with hydrazine or hydrazine derivatives. (Advances in Heterocyclic Chemistry; 1966, 6, 347):

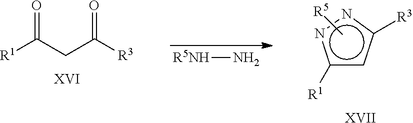

Alternatively 1,3-diketones could be prepared directly from ketones (XVIII) and acid chlorides (XIX) and then convert them in situ into pyrazoles by addition of hydrazine or hydrazine derivatives (Organic Letters 2006, 8, 13, 2675):

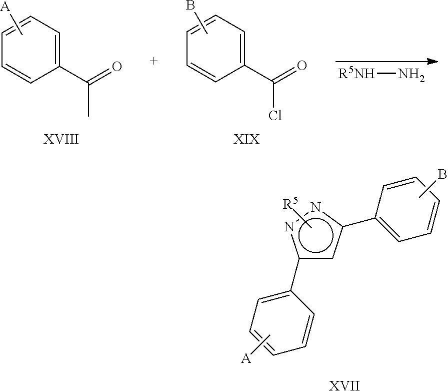

wherein A and B are any substituent in any position of the aryl ring.

Bromination of the intermediate XVII at C4 can be performed using conditions as described in Journal of Heterocyclic Chemistry 2006, 43, 1669:

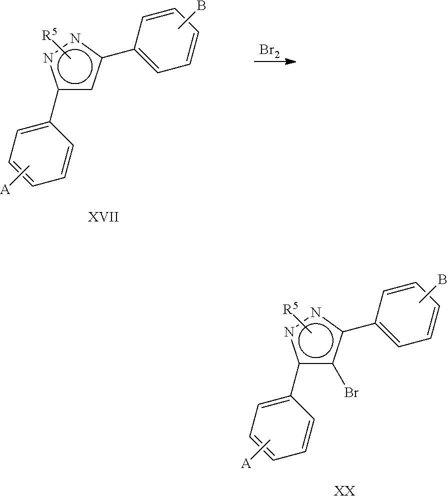

Metallation of the intermediate XX in position 4 followed by trapping with an aldehyde will lead to compounds of type XXI (Archiv der Pharmazie 1987, 320, 12, 1267):

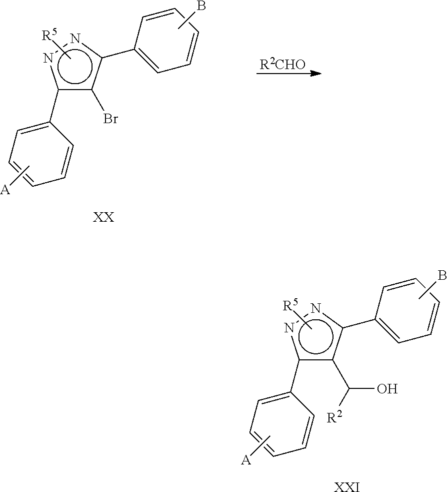

Alternatively Vilsmeier-Haack formylation on XVII will afford directly 4-formyl derivates XXII (Journal of Medicinal Chemistry; 2003, 46, 1144):

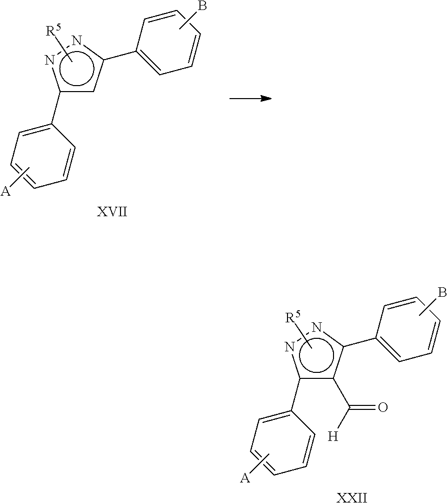

Intermediate XXII can further react with a Grignard or lithiated species to afford final compounds XXI:

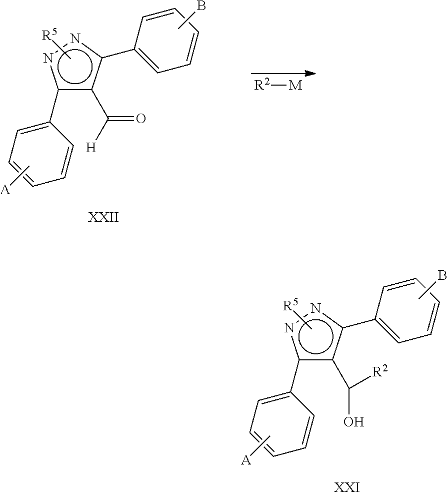

Other useful intermediates for the preparation of compounds of type Ib or Ic are found in the following journals and patent applications: Chemische Berichte, 1968, 101, 536:

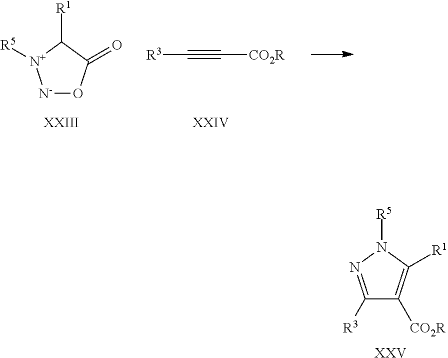

wherein R is an optionally substituted alkyl group.
Tetrahedron 2004, 60, 901:

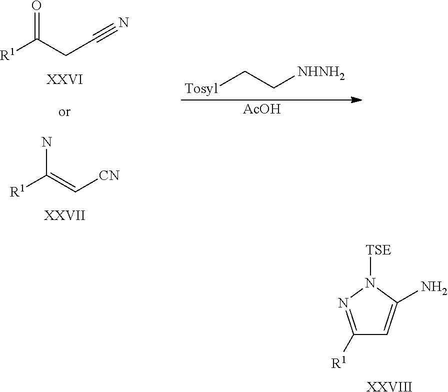

PCT Publication number WO 2006/092510:

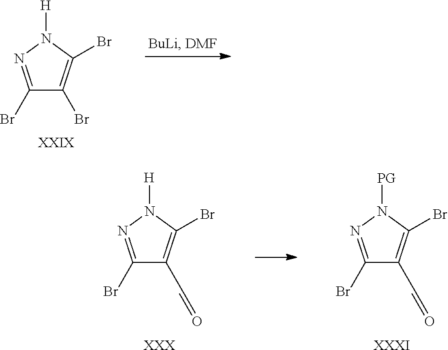

wherein PG is a protecting group.
Synlett 2004, 5, 795 and reference cited therein:

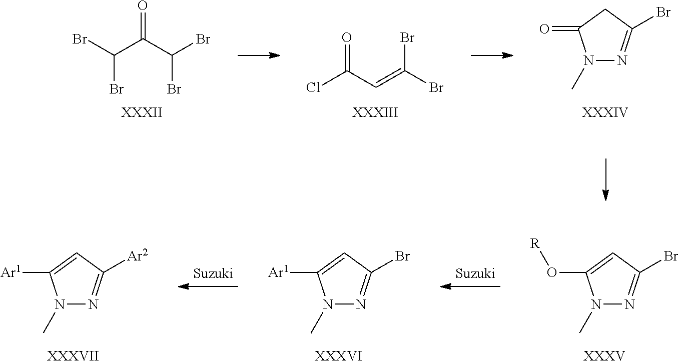

wherein R is CF₃SO₂ or C₄F₄SO₂ and Ar¹ and Ar² are optionally substituted aryl groups.

A review about the synthesis of pyrazoles can be found in Advances in Heterocyclic Chemistry 1990, 48, 223-99.

Examples of compounds of the present invention include, the following.

More particularly, compounds for use in the present invention are shown in Table I (compounds of formula (I)a), Table II (compounds of formula (I)b) and Table III (compounds of formula (I)c) below:

TABLE I
Ia

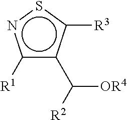

| No | R¹ | R² | R³ | R⁴ | Melting point or MS peak(s) |
|---|---|---|---|---|---|
| A1 | 4-Cl—Ph | 3-Py | Ph | H | 379/381 |
| A2 | 4-Cl—Ph | 3-Py | 4-Cl—Ph | H | 413/415 |
| A3 | 4-Cl—Ph | 3-Py | 4-Cl—Ph | C(O)Me | |
| A4 | 3-Cl—Ph | 3-Py | Ph | H | 379/381 |
| A5 | 4-Cl—Ph | 3-Py | 2-Cl—Ph | H | |
| A6 | 4-Cl—Ph | 3-Py | 5-Cl, 2-Thioph | H | |
| A7 | 4-Cl—Ph | 3-Py | 3-Cl—Ph | H | |
| A8 | 4-Cl—Ph | 3-Py | 5-Br, 2-Thioph | H | |
| A9 | 2,4-Cl₂—Ph | 3-Py | 3-Cl—Ph | C(O)Et | |
| A10 | 2,4-Cl₂—Ph | 3-Py | 4-Cl—Ph | H | |
| A11 | 2,4-Cl₂—Ph | 3-Py | Ph | H | |
| A12 | 2,4-Cl₂—Ph | 3-Py | 2-Cl—Ph | H | |
| A13 | 2,4-Cl₂—Ph | 3-Py | 3-Cl—Ph | H | |
| A14 | 4-Cl—Ph | 3-Py | 2,4-Cl₂—Ph | H | 447/449 |
| A15 | 2-Cl—Ph | 3-Py | 2,4-Cl₂—Ph | H | |
| A16 | 3-Cl—Ph | 3-Py | 2,4-Cl₂—Ph | H | 447/449 |
| A17 | 4-Cl—Ph | 5-Pyrimi | 4-Cl—Ph | H | |
| A18 | 4-Cl—Ph | 5-Pyrimi | 2-Cl—Ph | H | |
| A19 | 4-Cl—Ph | 5-Pyrimi | 3-Cl—Ph | H | |
| A20 | 2,4-Cl₂—Ph | 5-Pyrimi | 4-Cl—Ph | H | |
| A21 | 2,4-Cl₂—Ph | 5-Pyrimi | 2-Cl—Ph | H | |
| A22 | 2,4-Cl₂—Ph | 5-Pyrimi | 3-Cl—Ph | H | |
| A23 | 4-Cl—Ph | 5-Pyrimi | 2,4-Cl₂—Ph | H | |
| A24 | 2-Cl—Ph | 5-Pyrimi | 2,4-Cl₂—Ph | H | |
| A25 | 3-Cl—Ph | 5-Pyrimi | 2,4-Cl₂—Ph | H | |
| A26 | 2-F,4-Cl—Ph | 5-Pyrimi | 4-Cl—Ph | H | |
| A27 | 2-F,4-Cl—Ph | 5-Pyrimi | 2-Cl—Ph | H | |
| A28 | 2-F,4-Cl—Ph | 5-Pyrimi | 3-Cl—Ph | H | |
| A29 | 2-F,4-Cl—Ph | 3-Py | 4-Cl—Ph | H | |
| A30 | 2-F,4-Cl—Ph | 3-Py | 2-Cl—Ph | H | |
| A31 | 2-F,4-Cl—Ph | 3-Py | 3-Cl—Ph | H | |
| A32 | 2,4-F₂—Ph | 5-Pyrimi | 4-Cl—Ph | H | |
| A33 | 2,4-F₂—Ph | 5-Pyrimi | 2-Cl—Ph | H | |
| A34 | 2,4-F₂—Ph | 5-Pyrimi | 3-Cl—Ph | H | |
| A35 | 4-Cl—Ph | 4-F,3-Py | 2,4-Cl₂—Ph | H | |
| A36 | 2-Cl—Ph | 4-Me,3-Py | 2,4-Cl₂—Ph | H | |
| A37 | 3-Cl—Ph | 5-MeO,3-Py | 2,4-Cl₂—Ph | H | |
| A38 | 4-Cl—Ph | 3-Py | 2,4-Cl₂—Ph | C(O)^{c}Pr | |
| A39 | 2-Cl—Ph | 3-Py | 2,4-Cl₂—Ph | H | |
| A40 | 3-Cl—Ph | 3-Py | 5-Cl, 2-Thioph | H | |
| A41 | 4-Cl—Ph | 4-Me,5-Pyrimi | 4-Cl—Ph | H | |
| A42 | 4-Cl—Ph | 4-MeO,5-Pyrimi | 2-Cl—Ph | H | |
| A43 | 4-Cl—Ph | 5-Pyrimi | 3-CF₃—Ph | H | |
| A44 | 2-Thioph | 3-Py | 4-Cl—Ph | H | 172-173° C. |
| A45 | 4-Cl—Ph | 3-Py | 2-Thioph | H | |
| A46 | 2-Fur | 3-Py | 2,4-Cl₂-Ph | H | |
| A47 | 2,4-Cl₂—Ph | 3-Py | 2-Fur | H | |
| A48 | 3-Fur | 3-Py | 2,4-Cl₂—Ph | H | |
| A49 | 2,4-Cl₂—Ph | 3-Py | 3-Fur | H | |
| A50 | c-Hx | 3-Py | 4-Cl—Ph | H | |
| A51 | c-Hx | 3-Py | 2,4-F₂—Ph | H | |
| A52 | 2-Cl—Ph | 3-Py | c-Hx | H | |
| A53 | 2,4-Cl₂—Ph | 3-Py | c-Hx | H | |
| A54 | i-Pr | 3-Py | 4-Cl—Ph | H | |
| A55 | 2,4-Cl₂—Ph | 3-Py | i-Pr | H | |
| A56 | 2,4-Cl₂—Ph | 3-Py | i-Amyl | H | |
| A57 | 4-Cl—Ph | 3-Py | Et | H | |
| A58 | 4-Br—Ph | 3-Py | 4-Cl—Ph | H | |
| A59 | 2,4-Cl₂—Ph | 3-Py | 4-Cl—Ph | C(O)Me | |
| A60 | Bn | 3-Py | 4-Cl—Ph | H | |
| A61 | 2,4-Cl₂—Ph | 3-Py | 4-Br—Ph | H | |
| A62 | 2-Thioph | 3-Py | Ph | H | 351 |
| A63 | 2-Thioph | 3-Py | 2,4-Cl₂—Ph | H | 419/421 |
| A64 | 2-Thioph | 3-Py | 4-Br—Ph | H | 187-188° C. |
| A65 | 2-F—Ph | 3-Py | 4-Cl—Ph | H | 186-187° C. |
| A66 | 2-F—Ph | 3-Py | Ph | H | 363 |
| A67 | 2-F—Ph | 3-Py | 2,4-Cl₂—Ph | H | 182-183° C. |
| A68 | 2-F—Ph | 3-Py | 4-Br—Ph | H | 414/443 |
| A69 | 4-Cl—Ph | 3-Py | 4-Br—Ph | H | 457/459 |
| A70 | 4-Cl—Ph | 3-Py | 2-F,4-Cl—Ph | H | 431/433 |
| A71 | 2-Thioph | 3-Py | 2-F,4-Cl—Ph | H | 403/405 |
| A72 | 2-F—Ph | 3-Py | 2-F,4-Cl—Ph | H | 415/417 |
| A73 | 3-Cl—Ph | 3-Py | 4-Cl—Ph | H | 413/415 |
| A74 | 3-Cl—Ph | 3-Py | 4-Br—Ph | H | 457/459 |
| A75 | 3-Cl—Ph | 3-Py | 2-F,4-Cl—Ph | H | 431/433 |
| A76 | 2,4-F₂—Ph | 3-Py | Ph | H | 381/382 |
| A77 | 2,4-F₂—Ph | 3-Py | 4-Cl—Ph | H | 415/417 |
| A78 | 2,4-F₂-Ph | 3-Py | 4-Br—Ph | H | 459/461 |
| A79 | 2,4-F₂—Ph | 3-Py | 2,4-Cl₂—Ph | H | 449/451 |
| A80 | 2,4-F₂—Ph | 3-Py | 2-F,4-Cl—Ph | H | 433/435 |
| A81 | 2,4-F₂—Ph | 3-Pyrimi | 2-F,4-Cl—Ph | H | 184-185° C. |

Compounds A1 to A81 contain all one asymmetrical carbon atom which is the carbon atom linked to the OR⁴ and R² substituents.

TABLE II
Ib

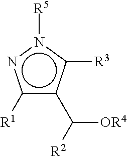

| | | | | | | Melting |
|---|---|---|---|---|---|---|
| | | | | | | point or MS |
| No | R¹ | R² | R³ | R⁴ | R⁵ | peak(s) |
| B1 | 4-Cl—Ph | 3-Py | Ph | H | H | |
| B2 | 4-Cl—Ph | 3-Py | 4-Cl—Ph | H | H | |
| B3 | 4-Cl—Ph | 3-Py | 4-Cl—Ph | C(O)Me | Me | |
| B4 | 3-Cl—Ph | 3-Py | Ph | H | Me | |
| B5 | 4-Cl—Ph | 3-Py | 2-Cl—Ph | H | Me | |
| B6 | 4-Cl—Ph | 3-Py | 5-Cl, 2-Thioph | H | Me | |
| B7 | 4-Cl—Ph | 3-Py | 3-Cl—Ph | H | Me | |
| B8 | 4-Cl—Ph | 3-Py | 5-Br, 2-Thioph | H | Me | |
| B9 | 2,4-Cl₂—Ph | 3-Py | 3-Cl—Ph | C(O)Et | Me | |
| B10 | 2,4-Cl₂—Ph | 3-Py | 4-Cl—Ph | H | Me | |
| B11 | 2,4-Cl₂—Ph | 3-Py | Ph | H | Me | |
| B12 | 2,4-Cl₂—Ph | 3-Py | 2-Cl—Ph | H | Me | |
| B13 | 2,4-Cl₂—Ph | 3-Py | 3-Cl—Ph | H | Me | |
| B14 | 4-Cl—Ph | 3-Py | 2,4-Cl₂—Ph | H | Me | |
| B15 | 2-Cl—Ph | 3-Py | 2,4-Cl₂—Ph | H | Me | |
| B16 | 3-Cl—Ph | 3-Py | 2,4-Cl₂—Ph | H | Me | |
| B17 | 4-Cl—Ph | 5-Pyrimi | 4-Cl—Ph | H | Me | |
| B18 | 4-Cl—Ph | 5-Pyrimi | 2-Cl—Ph | H | Me | |
| B19 | 4-Cl—Ph | 5-Pyrimi | 3-Cl—Ph | H | Me | |
| B20 | 2,4-Cl₂—Ph | 5-Pyrimi | 4-Cl—Ph | H | Me | |
| B21 | 2,4-Cl₂—Ph | 5-Pyrimi | 2-Cl—Ph | H | Me | |
| B22 | 2,4-Cl₂—Ph | 5-Pyrimi | 3-Cl—Ph | H | Me | |
| B23 | 4-Cl—Ph | 5-Pyrimi | 2,4-Cl₂—Ph | H | Me | |
| B24 | 2-Cl—Ph | 5-Pyrimi | 2,4-Cl₂—Ph | H | Me | |
| B25 | 3-Cl—Ph | 5-Pyrimi | 2,4-Cl₂—Ph | H | Me | |
| B26 | 2-F,4-Cl—Ph | 5-Pyrimi | 4-Cl—Ph | H | Me | |
| B27 | 2-F,4-Cl—Ph | 5-Pyrimi | 2-Cl—Ph | H | Me | |
| B28 | 2-F,4-Cl—Ph | 5-Pyrimi | 3-Cl—Ph | H | Me | |
| B29 | 2-F,4-Cl—Ph | 3-Py | 4-Cl—Ph | H | Me | |
| B30 | 2-F,4-Cl—Ph | 3-Py | 2-Cl—Ph | H | Me | |
| B31 | 2-F,4-Cl—Ph | 3-Py | 3-Cl—Ph | H | Me | |
| B32 | 2,4-F₂—Ph | 5-Pyrimi | 4-Cl—Ph | H | Me | |
| B33 | 2,4-F₂—Ph | 5-Pyrimi | 2-Cl—Ph | H | Me | |
| B34 | 2,4-F₂—Ph | 5-Pyrimi | 3-Cl—Ph | H | Me | |
| B35 | 4-Cl—Ph | 4-F,3-Py | 2,4-Cl₂—Ph | H | Me | |
| B36 | 2-Cl—Ph | 4-Me,3-Py | 2,4-Cl₂—Ph | H | Me | |
| B37 | 3-Cl—Ph | 5-MeO,3-Py | 2,4-Cl₂—Ph | H | Me | |
| B38 | 4-Cl—Ph | 3-Py | 2,4-Cl₂—Ph | C(O)^{c}Pr | Me | |
| B39 | 2-Cl—Ph | 3-Py | 2,4-Cl₂—Ph | H | Me | |
| B40 | 3-Cl—Ph | 3-Py | 5-Cl, 2-Thioph | H | Me | |
| B41 | 4-Cl—Ph | 4-Me,5-Pyrimi | 4-Cl-Ph | H | Me | |
| B42 | 4-Cl—Ph | 4-MeO,5-Pyrimi | 2-Cl-Ph | H | Me | |
| B43 | 4-Cl—Ph | 5-Pyrimi | 3-CF₃—Ph | H | Me | |
| B44 | 2-Thioph | 3-Py | 4-Cl—Ph | H | Me | |
| B45 | 4-Cl—Ph | 3-Py | 2-Thioph | H | Me | |
| B46 | 2-Fur | 3-Py | 2,4-Cl₂—Ph | H | Me | |
| B47 | 2,4-Cl₂—Ph | 3-Py | 2-Fur | H | Me | |
| B48 | 3-Fur | 3-Py | 2,4-Cl₂—Ph | H | Me | |
| B49 | 2,4-Cl₂—Ph | 3-Py | 3-Fur | H | Me | |
| B50 | c-Hx | 3-Py | 4-Cl—Ph | H | Me | |
| B51 | c-Hx | 3-Py | 2,4-F₂—Ph | H | Me | |
| B52 | 2-Cl—Ph | 3-Py | c-Hx | H | Me | |
| B53 | 2,4-Cl₂—Ph | 3-Py | c-Hx | H | Me | |
| B54 | i-Pr | 3-Py | 4-Cl—Ph | H | Me | |
| B55 | 2,4-Cl₂—Ph | 3-Py | i-Pr | H | Me | |
| B56 | 2,4-Cl₂—Ph | 3-Py | i-Amyl | H | Me | |
| B57 | 4-Cl—Ph | 3-Py | Et | H | Me | |
| B58 | 4-Br—Ph | 3-Py | 4-Cl—Ph | H | Me | |
| B59 | 2,4-Cl₂—Ph | 3-Py | 4-Cl—Ph | C(O)Me | Me | |
| B60 | Bn | 3-Py | 4-Cl—Ph | H | Me | |
| B61 | 2,4-Cl₂—Ph | 3-Py | 4-Br—Ph | H | Me | |
| B62 | 4-Cl—Ph | 3-Py | Ph | H | Me | |
| B63 | 4-Cl—Ph | 3-Py | Ph | H | Ph | |
| B64 | 4-Cl—Ph | 3-Py | 4-Cl—Ph | C(O)Me | Et | |
| B65 | 4-Cl—Ph | 3-Py | 3-Cl—Ph | H | H | |
| B66 | 4-Cl—Ph | 3-Py | 3-Cl—Ph | H | Et | |
| B67 | 4-Cl—Ph | 3-Py | 3-Cl—Ph | H | c-Pr | |
| B68 | 3-Cl—Ph | 3-Py | 4-Cl—Ph | H | H | |
| B69 | 4-Cl—Ph | 3-Py | 3-Cl—Ph | H | i-Pr | |
| B70 | 3-Cl—Ph | 3-Py | 4-Cl—Ph | H | Me | |
| B71 | 3-Cl—Ph | 3-Py | 4-Cl—Ph | H | Ph | |
| B72 | 2,4-Cl₂—Ph | 3-Py | 4-Cl—Ph | H | H | |
| B73 | 2,4-Cl₂—Ph | 3-Py | Ph | H | H | |
| B74 | 2,4-Cl₂—Ph | 3-Py | 2-Cl—Ph | H | H | |
| B75 | 2,4-Cl₂—Ph | 3-Py | 3-Cl—Ph | H | H | |
| B76 | 2-F,4-Cl—Ph | 5-Pyrimi | 4-Cl—Ph | H | H | |
| B77 | 2-F,4-Cl—Ph | 5-Pyrimi | 2-Cl—Ph | H | H | |
| B78 | 2-F,4-Cl—Ph | 5-Pyrimi | 3-Cl—Ph | H | H | |
| B79 | 2-F,4-Cl—Ph | 3-Py | 4-Cl—Ph | H | H | |
| B80 | 4-Cl—Ph | 5-Pyrimi | 2-F,4-Cl—Ph | H | H | |
| B81 | 2-Cl—Ph | 5-Pyrimi | 2-F,4-Cl—Ph | H | H | |
| B82 | 3-Cl—Ph | 5-Pyrimi | 2-F,4-Cl—Ph | H | H | |
| B83 | 4-Cl—Ph | 3-Py | 2-F,4-Cl—Ph | H | H | |
| B84 | 4-Br—Ph | 3-Py | 4-OMe—Ph | H | Me | 450/452 |
| B85 | 4-Cl—Ph | 3-Py | 4-Cl—Ph | H | Me | 410/412 |
| B86 | 2,4-F₂—Ph | 3-Py | 2,4-F₂—Ph | H | Me. | 199-200° C |
| B87 | 4-Cl—Ph | 3-Py | 2,4-F₂—Ph | H | Me | 117-119° C. |
| B88 | 4-F—Ph | 3-Py | 4-Cl—Ph | H | Me | 394/396 |
| B89 | 3-F—Ph | 3-Py | 4-Cl—Ph | H | Me | 394/396 |
| B90 | 2-F—Ph | 3-Py | 4-Cl—Ph | H | Me | 394/396 |
| B91 | 4-Cl—Ph | 3-Py | 4-Cl—Ph | H | Bn | 486/488 |

Compounds B1 to B91 contain all one asymmetrical carbon atom which is the carbon atom linked to the OR⁴ and R² substituents.

TABLE III
Ic

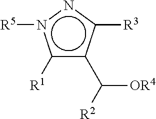

| | | | | | | Melting point |
|---|---|---|---|---|---|---|
| No | R¹ | R² | R³ | R⁴ | R⁵ | or MS peak(s) |
| C1 | 4-Cl—Ph | 3-Py | Ph | H | H | |
| C2 | 4-Cl—Ph | 3-Py | 4-Cl—Ph | H | H | |
| C3 | 4-Cl—Ph | 3-Py | 4-Cl—Ph | C(O)Me | Me | |
| C4 | 3-Cl—Ph | 3-Py | Ph | H | Me | |
| C5 | 4-Cl—Ph | 3-Py | 2-Cl—Ph | H | Me | |
| C6 | 4-Cl—Ph | 3-Py | 5-Cl, 2- | H | Me | |
| | | | Thioph | | | |
| C7 | 4-Cl—Ph | 3-Py | 3-Cl—Ph | H | Me | |
| C8 | 4-Cl—Ph | 3-Py | 5-Br, 2- | H | Me | |
| | | | Thioph | | | |
| C9 | 2,4-Cl₂—Ph | 3-Py | 3-Cl—Ph | C(O)Et | Me | |
| C10 | 2,4-Cl₂—Ph | 3-Py | 4-Cl—Ph | H | Me | |
| C11 | 2,4-Cl₂—Ph | 3-Py | Ph | H | Me | |
| C12 | 2,4-Cl₂—Ph | 3-Py | 2-Cl—Ph | H | Me | |
| C13 | 2,4-Cl₂—Ph | 3-Py | 3-Cl—Ph | H | Me | |
| C14 | 4-Cl—Ph | 3-Py | 2,4-Cl₂—Ph | H | Me | |
| C15 | 2-Cl—Ph | 3-Py | 2,4-Cl₂—Ph | H | Me | |
| C16 | 3-Cl—Ph | 3-Py | 2,4-Cl₂—Ph | H | Me | |
| C17 | 4-Cl—Ph | 5-Pyrimi | 4-Cl—Ph | H | Me | |
| C18 | 4-Cl—Ph | 5-Pyrimi | 2-Cl—Ph | H | Me | |
| C19 | 4-Cl—Ph | 5-Pyrimi | 3-Cl—Ph | H | Me | |
| C20 | 2,4-Cl₂—Ph | 5-Pyrimi | 4-Cl—Ph | H | Me | |
| C21 | 2,4-Cl₂—Ph | 5-Pyrimi | 2-Cl—Ph | H | Me | |
| C22 | 2,4-Cl₂—Ph | 5-Pyrimi | 3-Cl—Ph | H | Me | |
| C23 | 4-Cl—Ph | 5-Pyrimi | 2,4-Cl₂—Ph | H | Me | |
| C24 | 2-Cl—Ph | 5-Pyrimi | 2,4-Cl₂—Ph | H | Me | |
| C25 | 3-Cl—Ph | 5-Pyrimi | 2,4-Cl₂—Ph | H | Me | |
| C26 | 2-F,4-Cl—Ph | 5-Pyrimi | 4-Cl—Ph | H | Me | |
| C27 | 2-F,4-Cl—Ph | 5-Pyrimi | 2-Cl—Ph | H | Me | |
| C28 | 2-F,4-Cl—Ph | 5-Pyrimi | 3-Cl—Ph | H | Me | |
| C29 | 2-F,4-Cl—Ph | 3-Py | 4-Cl—Ph | H | Me | |
| C30 | 2-F,4-Cl—Ph | 3-Py | 2-Cl—Ph | H | Me | |
| C31 | 2-F,4-Cl—Ph | 3-Py | 3-Cl—Ph | H | Me | |
| C32 | 2,4-F₂—Ph | 5-Pyrimi | 4-Cl—Ph | H | Me | |
| C33 | 2,4-F₂—Ph | 5-Pyrimi | 2-Cl—Ph | H | Me | |
| C34 | 2,4-F₂—Ph | 5-Pyrimi | 3-Cl—Ph | H | Me | |
| C35 | 4-Cl—Ph | 4-F,3-Py | 2,4-Cl₂—Ph | H | Me | |
| C36 | 2-Cl—Ph | 4-Me,3-Py | 2,4-Cl₂—Ph | H | Me | |
| C37 | 3-Cl—Ph | 5-MeO,3-Py | 2,4-Cl₂—Ph | H | Me | |
| C38 | 4-Cl—Ph | 3-Py | 2,4-Cl₂—Ph | C(O)^{c}Pr | Me | |
| C39 | 2-Cl—Ph | 3-Py | 2,4-Cl₂—Ph | H | Me | |
| C40 | 3-Cl—Ph | 3-Py | 5-Cl, 2- | H | Me | |
| | | | Thioph | | | |
| C41 | 4-Cl—Ph | 4-Me,5-Pyrimi | 4-Cl—Ph | H | Me | |
| C42 | 4-Cl—Ph | 4-MeO,5-Pyrimi | 2-Cl—Ph | H | Me | |
| C43 | 4-Cl—Ph | 5-Pyrimi | 3-CF₃—Ph | H | Me | |
| C44 | 2-Thioph | 3-Py | 4-Cl—Ph | H | Me | |
| C45 | 4-Cl—Ph | 3-Py | 2-Thioph | H | Me | |
| C46 | 2-Fur | 3-Py | 2,4-Cl₂—Ph | H | Me | |
| C47 | 2,4-Cl₂—Ph | 3-Py | 2-Fur | H | Me | |
| C48 | 3-Fur | 3-Py | 2,4-Cl₂—Ph | H | Me | |
| C49 | 2,4-Cl₂—Ph | 3-Py | 3-Fur | H | Me | |
| C50 | c-Hx | 3-Py | 4-Cl—Ph | H | Me | |
| C51 | c-Hx | 3-Py | 2,4-F₂—Ph | H | Me | |
| C52 | 2-Cl—Ph | 3-Py | c-Hx | H | Me | |
| C53 | 2,4-Cl₂—Ph | 3-Py | c-Hx | H | Me | |
| C54 | i-Pr | 3-Py | 4-Cl—Ph | H | Me | |
| C55 | 2,4-Cl₂—Ph | 3-Py | i-Pr | H | Me | |
| C56 | 2,4-Cl₂—Ph | 3-Py | i-Amyl | H | Me | |
| C57 | 4-Cl—Ph | 3-Py | Et | H | Me | |
| C58 | 4-Br—Ph | 3-Py | 4-Cl—Ph | H | Me | |
| C59 | 2,4-Cl₂—Ph | 3-Py | 4-Cl—Ph | C(O)Me | Me | |
| C60 | Bn | 3-Py | 4-Cl—Ph | H | Me | |
| C61 | 2,4-Cl₂—Ph | 3-Py | 4-Br—Ph | H | Me | |
| C62 | 4-Cl—Ph | 3-Py | Ph | H | Me | |
| C63 | 4-Cl—Ph | 3-Py | Ph | H | Ph | |
| C64 | 4-Cl—Ph | 3-Py | 4-Cl—Ph | C(O)Me | Et | |
| C65 | 4-Cl—Ph | 3-Py | 3-Cl—Ph | H | H | |
| C66 | 4-Cl—Ph | 3-Py | 3-Cl—Ph | H | Et | |
| C67 | 4-Cl—Ph | 3-Py | 3-Cl—Ph | H | c-Pr | |
| C68 | 3-Cl—Ph | 3-Py | 4-Cl—Ph | H | H | |
| C69 | 4-Cl—Ph | 3-Py | 3-Cl—Ph | H | i-Pr | |
| C70 | 3-Cl—Ph | 3-Py | 4-Cl—Ph | H | Me | |
| C71 | 3-Cl—Ph | 3-Py | 4-Cl—Ph | H | Ph | |
| C72 | 2,4-Cl₂—Ph | 3-Py | 4-Cl—Ph | H | H | |
| C73 | 2,4-Cl₂—Ph | 3-Py | Ph | H | H | |
| C74 | 2,4-Cl₂—Ph | 3-Py | 2-Cl—Ph | H | H | |
| C75 | 2,4-Cl₂—Ph | 3-Py | 3-Cl—Ph | H | H | |
| C76 | 2-F,4-Cl—Ph | 5-Pyrimi | 4-Cl—Ph | H | H | |
| C77 | 2-F,4-Cl—Ph | 5-Pyrimi | 2-Cl—Ph | H | H | |
| C78 | 2-F,4-Cl—Ph | 5-Pyrimi | 3-Cl—Ph | H | H | |
| C79 | 2-F,4-Cl—Ph | 3-Py | 4-Cl—Ph | H | H | |
| C80 | 4-Cl—Ph | 5-Pyrimi | 2-F,4-Cl—Ph | H | H | |
| C81 | 2-Cl—Ph | 5-Pyrimi | 2-F,4-Cl—Ph | H | H | |
| C82 | 3-Cl—Ph | 5-Pyrimi | 2-F,4-Cl—Ph | H | H | |
| C83 | 4-Cl—Ph | 3-Py | 2-F,4-Cl—Ph | H | H | |
| C84 | 2,4-F₂—Ph | 3-Py | 4-Cl—Ph | H | Me | 190-194° C. |
| C85 | 4-Cl—Ph | 3-Py | 4-F—Ph | H | Me | 394/396 |
| C86 | 3-F—Ph | 3-Py | 4-Cl—Ph | H | Me | 394/396 |
| C87 | 2-F—Ph | 3-Py | 4-Cl—Ph | H | Me | 394/396 |

Compounds C1 to C87 contain all one asymmetrical carbon atom which is the carbon atom linked to the OR⁴ and R² substituents.
In the above tables, the following is meant by each abbreviation given for R¹ to R⁵:

| | | | |
|---|---|---|---|
| H | Hydrogen | 4-Br—Ph | 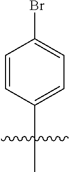 |
| Me | 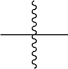 | 2-Fl—Ph | 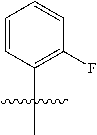 |
| Et | 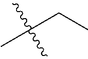 | 2-Cl—Ph | 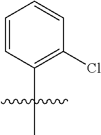 |
| i-Pr | 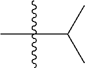 | 3-Cl—Ph | 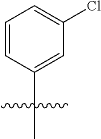 |
| i-Amyl | 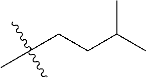 | 4-Cl—Ph | 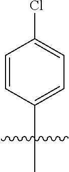 |
| C(O)Me | 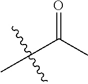 | 2,4-Cl₂—Ph | 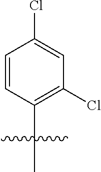 |
| C(O)Et | 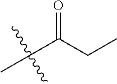 | 2-F,4-Cl—Ph | 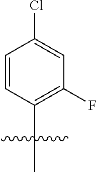 |
| C(O)^{c}Pr | 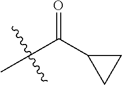 | 2,4-F₂—Ph | 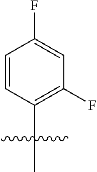 |
| c-Hx | 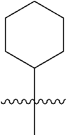 | 3-CF₃—Ph | 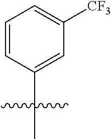 |
| Bn | 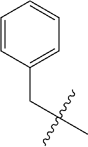 | 4-F,3-Py | 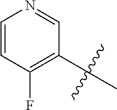 |
| Ph | 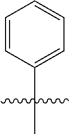 | 4-Me,3-Py | 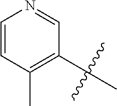 |
| 3-Py | 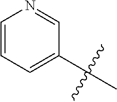 | 5-MeO,3-Py | 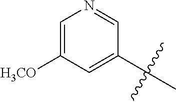 |
| 5-Pyrimi | 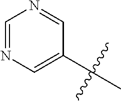 | 4-Me,5-Pyrimi | 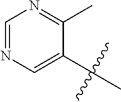 |
| 2-Fur | 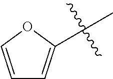 | 4-MeO,5-Pyrimi | 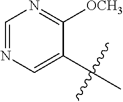 |
| 3-Fur | 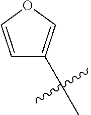 | 5-Cl, 2-Thioph | 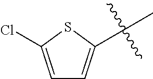 |
| 2-Thioph | 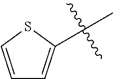 | 5-Br, 2-Thioph | 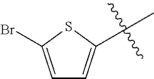 |

The isothiazole and pyrazole of the formula (I) can be obtained in different tautomeric forms.

The present invention will now be described by way of the following examples.

### EXAMPLES

### Example 1

### Preparation of [3-(4-Chloro-phenyl)-5-phenyl-isothiazol-4-yl]-pyridin-3-yl-methanol (compound A1; see Table I)

### Step a: 5-(4-Chloro-phenyl)-[1,3,4]oxathiazol-2-one (3)

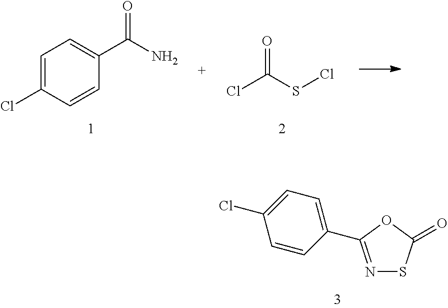

To a suspension of 1 (778 mg) in 10 ml of toluene is added 0.85 ml of chlorocarbonylsulfenyl chloride and the mixture is heated at 100° C. for 2 h. Gas evolution is observed and a clear solution is obtained. TLC shows complete conversion. The reaction mixture is concentrated and the solid residue is triturated with pentane, filtered and dried. Yield: 886 mg (82%) of 3 as a white crystalline solid.

### Step b: 3-(4-Chloro-phenyl)-isothiazole-4,5-dicarboxylic acid dimethyl ester (5)

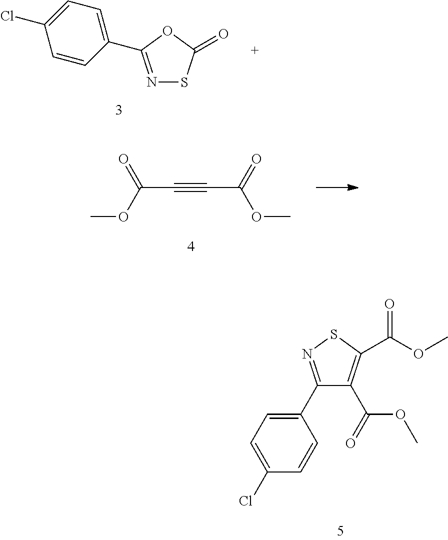

To a solution of 3 (1.068 g) in 10 ml of α,α,α-trifluorotoluene is added 2.0 ml of dimethyl acetylenedicarboxylate and the mixture is heated in the microwave at 170° C. for 1 h. GCMS shows complete conversion into product 5.

The reaction mixture is concentrated and the oily residue (containing excess 4) is purified by flash column chromatography (20% EtOAc in heptane). The fractions containing product are combined and concentrated. Reactant 4 is still present. It is successfully removed by addition of pentane and taking of the solvent with a pipette. This process is repeated four times. Compound 5 is obtained as a white solid with a melting point: 106.5 to 107.5° C.

### Step c: 3-(4-Chloro-phenyl)-isothiazole-4,5-dicarboxylic acid (6)

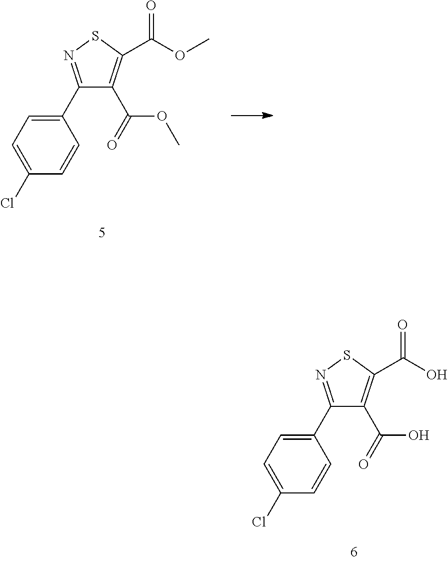

A solution of 4.4 g of 5 and 2.8 g of NaOH in 20 ml of water is kept at reflux for 2.5 h. The reaction mixture is cooled, diluted with water (150 ml) and acidified with conc. HCl (aq). A precipitate forms. The water layer is extracted with EtOAc (2×200 ml; the precipitate slowly dissolves). The combined organic layers are washed with brine and dried (Na₂SO₄). Concentration afforded 3.9 grams of 6 as a white solid.

### Step d: 3-(4-Chloro-phenyl)-isothiazole-4-carboxylic acid (7)

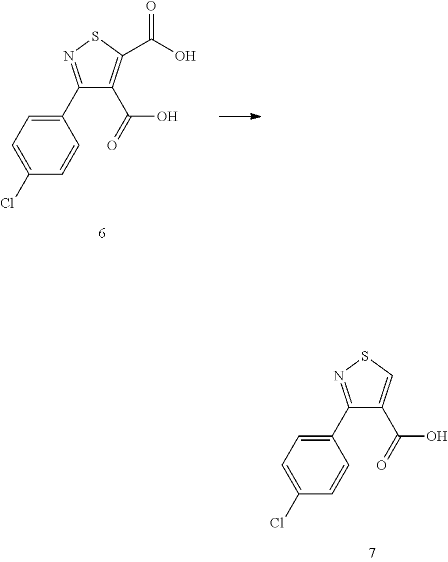

A suspension of 6 (3.9) g in 40 ml of 1,2-dichlorobenzene is held at reflux for 20 min (gas formation is observed). The reaction mixture is cooled (precipitation of product), diluted with pentane (50 ml) and filtered. The cream colored solid is washed with pentane (5×) and dried. This product is still contaminated with 1,2-dichlorobenzene. The crude product is suspended in water (80 ml) and 20 ml 1N NaOH is added, a clear solution results. The water layer is extracted with ether (2×100 ml). The clear water layer is acidified with conc. HCl until pH 2 to 3 (precipitation of product). The product is extracted with EtOAc (2×100 ml). The combined organic layers are washed with brine, dried (Na₂SO₄) and concentrated. Yield: 3.1 g of 7 as an off white solid (melting point: 179.5-180.5° C.).

### Step e: 3-(4-Chloro-phenyl)-isothiazole-4-carboxylic acid tert-butyl ester (8)

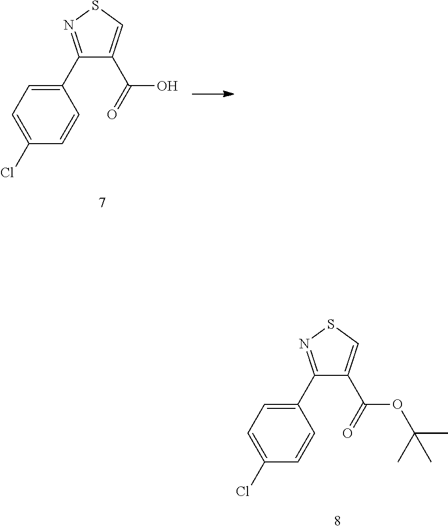

To a mixture of 7 (3.35 g), DMAP (1.7 g) and tBuOH (5.2 g) in CH₂Cl₂ (50 ml) is added 3.2 g of EDCI and the clear solution is stirred over the weekend. Additional CH₂Cl₂ (100 ml) is added and the mixture is washed with 1N HCl (2×150 ml), sat. NaHCO₃ (150 ml), brine (150 ml), dried and concentrated. This afforded 3.75 g of 8 as a pale yellow oil.

### Step f: 5-Bromo-3-(4-chloro-phenyl)-isothiazole-4-carboxylic acid tert-butyl ester (9)

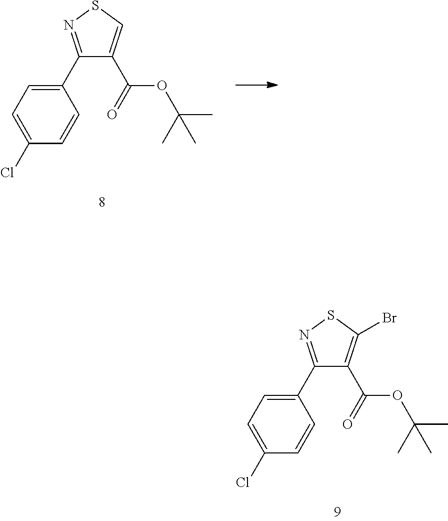

A solution of 8 (3.50 g) in THF (60 ml) is cooled to −78° C. under N₂ followed by drop wise addition of BuLi (8.0 ml, 1.6M in hexane). After complete addition stirring is continued at −78° C. for 15 min. Bromine (1.2 ml) is now added drop wise and stirring is continued at −78° C. for 15 min, after which the cooling bath is removed. The mixture is allowed to warm to room temp. TLC shows a good conversion into a slightly faster moving spot (Hep/EA, 9/1). 1N HCl (50 ml) is added and the THF is removed in vacuo. Water (100 ml), containing some sodium thiosulfate, is added and the product is extracted with EtOAc (150 ml). The organic layer is washed with sat. NaHCO₃, brine, dried and concentrated. The orange/red oily residue is purified by flash column chromatography (˜50 g silica, 5% diisopropyl ether in heptane) yielding 3.1 g of 9 as a pale yellow oil.

### Step g: 3-(4-Chloro-phenyl)-5-phenyl-isothiazole-4-carboxylic acid tert-butylester (11)

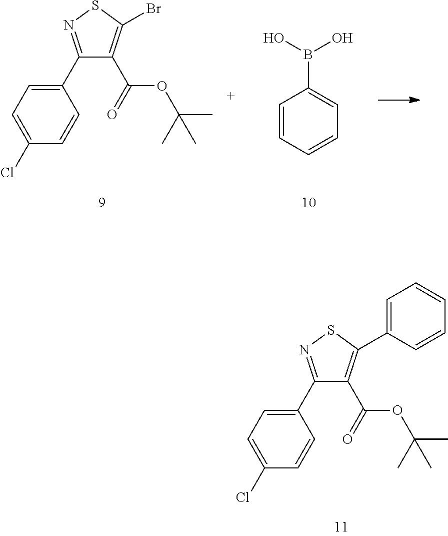

Starting material 9 (1.12 g) is dissolved in DME (12 ml) and to this are added 439 mg of 10, water (5 ml) and 954 mg of Na₂CO₃. The mixture is degassed with argon for 5 min. Pd(dppf)Cl₂ (121 mg) is added, the tube is sealed and the reaction mixture is heated at 100° C. in the microwave for 15 min. TLC (Hep/EtOAc, 80/20) shows a good conversion into a slower moving spot. The reaction mixture is diluted with EtOAc and washed with water and brine. The organic layer is dried and concentrated. The residue is purified by flash column chromatography using 5% diisopropyl ether in heptane as eluent. Isothiazole 11 is obtained in good yield as a white crystalline solid.

### Step h: 3-(4-Chloro-phenyl)-5-phenyl-isothiazole-4-carboxylic acid (12)

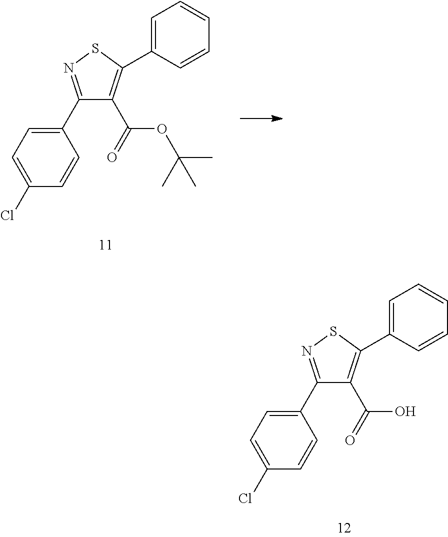

To a solution of 11 (2.35 g) in CH₂Cl₂ (25 ml) is added 25 ml of TFA and the mixture is stirred at room temperature for 24 h. The mixture is then concentrated and the residue is dissolved in ether (250 ml). Water (225 ml) is added followed by 1N NaOH (25 ml). After washing and separation, the basic water layer is extracted once more with ether. The water layer is acidified with conc. HCl and the precipitated product is extracted with ether (2×150 ml). The combined organic layers are washed with brine, dried and concentrated. 1.85 g of 12 is obtained as a cream colored solid.

### Step i: 3-(4-Chlorophenyl)-5-phenylisothiazole-4-carboxylic acid methoxymethylamide (14)

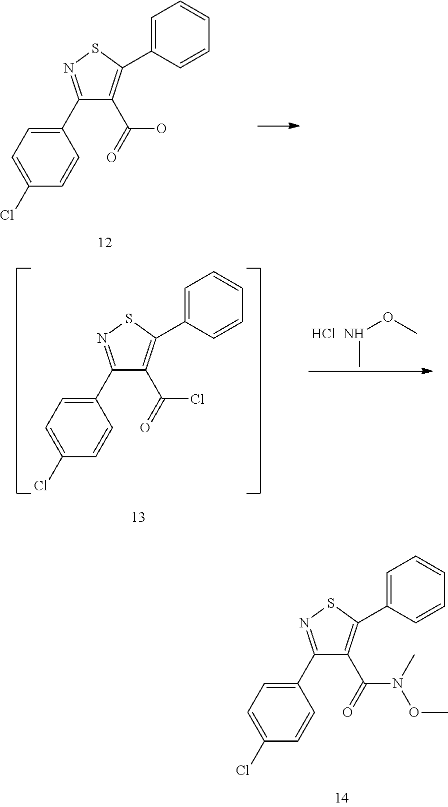

To a suspension of 12 (700 mg) in CH₂Cl₂ (10 ml) is added oxalyl chloride (0.76 ml) followed by one drop of DMF. A vigorous reaction took place. After stirring for several minutes a clear solution is obtained, which is stirred overnight. The reaction mixture is concentrated and stripped with toluene (2×). The yellow solid is dissolved in CH₂Cl₂ (10 ml), O,N-Dimethyl-hydroxylamine hydrochloride (432 mg) is added followed by the addition of 1.5 ml of Et₃N. After stirring for 1 h the mixture is concentrated. The solid residue is partitioned between EtOAc and 1N HCl. After washing, the layers are separated and the organic layer is washed once more with 1 N HCl, sat. NaHCO₃, brine, dried and concentrated. The residue is purified by flash column chromatography (20% EtOAc in heptane). 667 mg of 14 is obtained as a white crystalline solid with a melting point of 119-120° C.

### Step k: 3-(4-Chloro-phenyl)-5-phenyl-isothiazole-4-carbaldehyde (15)

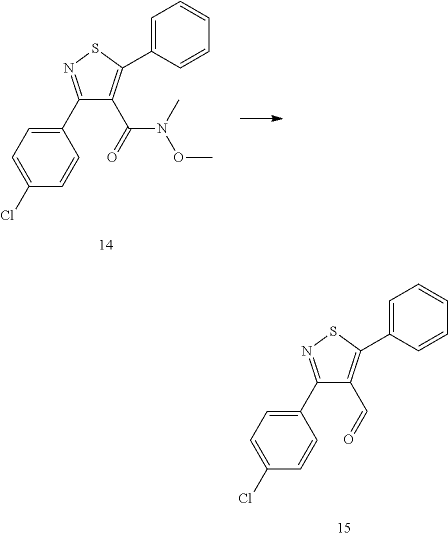

A solution of 14 (359 mg) in 4 ml of THF is cooled to −78° C. under N₂ and to this is added 1.5 ml of DIBALH (1M in hexane). After stirring for 30 min at −78° C., only starting material is observed. The iPrOH/CO₂ bath is replaced for an ice bath and stirring is continued at 0° C. for 2 h. TLC shows complete conversion. The mixture is re-cooled to −78° C. and the reaction mixture is quenched with 1N HCl (5 ml). The cooling bath is removed and the reaction mixture is diluted with 1N HCl (50 ml) and EtOAc (50 ml). The layers are separated and the organic layer is washed once more with 1N HCl (50 ml). The organic layer is washed with brine, dried and concentrated. 286 mg of 15 are obtained as a white solid with melting point: 148-149° C.

### Step l: [3-(4-Chloro-phenyl)-5-phenyl-isothiazol-4-yl]-pyridin-3-yl-methanol (16)

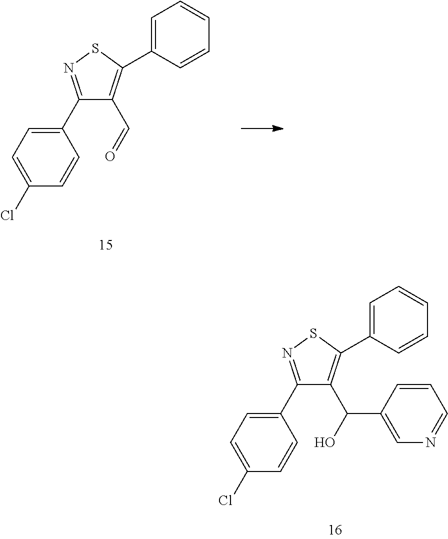

A solution of 3-bromopyridine (103 mg) in 2.5 ml of ether is cooled to −78° C. under N₂ and to this is added drop-wise a BuLi solution (0.375 ml, 1.6 M in hex). After stirring for 15 min, a solution of 15 (150 mg) in THF is added drop-wise. Stirring is continued for 20 min at −78° C., the red colored reaction mixture is quenched with water (5 ml) and the cooling bath is removed. Water (25 ml) and EtOAc (30 ml) are added. After washing, the layers are separated, the organic layer is washed with brine, dried and concentrated to afford an orange foam (200 mg). The material is purified by flash column chromatography (2% MeOH in CH₂Cl₂). Desired compound 16 is obtained as white foam showing the expected mass in LCMS (M⁺¹:379 and its isotopes at 380; 381 and 382).

### Example 2

### Preparation of [3-(4-Chloro-phenyl)-5-(2,4-difluoro-phenyl)-1-methyl-1H-pyrazol-4-yl]-pyridin-3-yl-methanol (compound B87; see Table II)

### Step a: Mixture of 3-(4-Chloro-phenyl)-5-(2,4-difluoro-phenyl)-1-methyl-1H-pyrazole (19) and 5-(4-Chloro-phenyl)-3-(2,4-difluoro-phenyl)-1-methyl-1H-pyrazole (20)

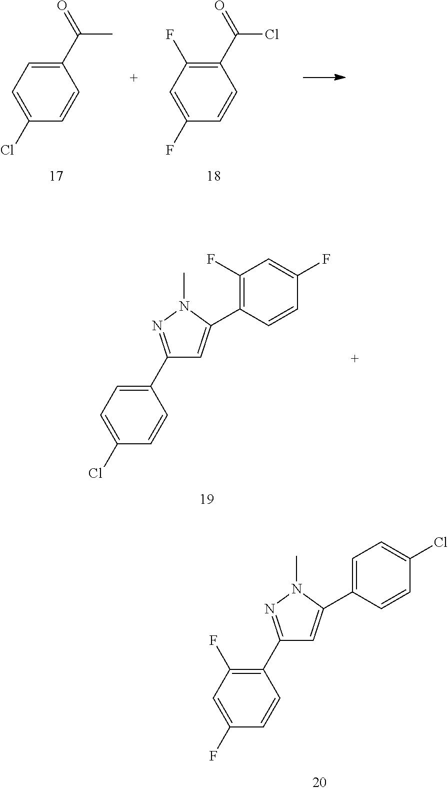

4-Chloroacetophenone (4 g) is dissolved in toluene (50 ml) and the solution is cooled to 0° C. under nitrogen. Lithium-bis(trimethylsilyl)amide solution (27.2 ml, 1M in THF) is added quickly via syringe with stirring, and the formed anion is allowed to stir for approximately 10 minutes before the addition of 2,4-difluorobenzoyl chloride (2.28 g) in one portion. The reaction mixture is removed from ice-bath and allowed to stand for 5 minutes. Acetic acid (20 ml) is added followed by EtOH (100 ml) and THF (50 ml) and finally methyl hydrazine (20.4 g). The resulting solution is added to NaOH 1M solution and extracted twice with EtOAc. The combined organic layers are washed with brine, dried and concentrated to afford a crude material that is purified by flash column chromatography (30% EtOAc in cyclohexane). This afforded an inseparable mixture of (19) and (20), in a ratio 1:1, as a pale yellow oil (3.3 g).

### Step b: Preparation of 3-(4-Chloro-phenyl)-5-(2,4-difluoro-phenyl)-1-methyl-1H-pyrazole-4-carbaldehyde (23) and 5-(4-Chloro-phenyl)-3-(2,4-difluoro-phenyl)-1-methyl-1H-pyrazole-4-carbaldehyde (24)

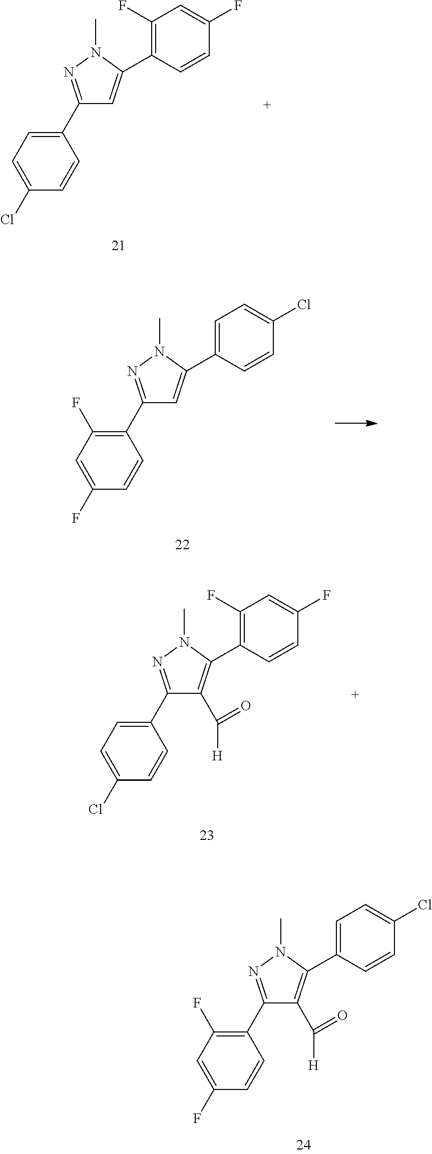

The mixture above (2 g) is dissolved in DMF (3.1 ml) and phosphorus pentachloride (2 g) is added. The reaction is stirred overnight at 70° C. The reaction is cooled to room temperature, diluted with water and neutralized with a saturated solution of Na₂CO₃. The aqueous layer is extracted with DCM (3×50 ml), dried and concentrated in vacuo. The residue is purified by flash column chromatography (20% EtOAc in cyclohexane). The desired compounds, (23) and (24) are isolated as a foam (0.41 g) and as a white crystalline solid (0.43 g) respectively.

### Step c: [3-(4-Chloro-phenyl)-5-(2,4-difluoro-phenyl)-1-methyl-1H-pyrazol-4-yl]-pyridin-3-yl-methanol (25) (compound B87 see Table II)

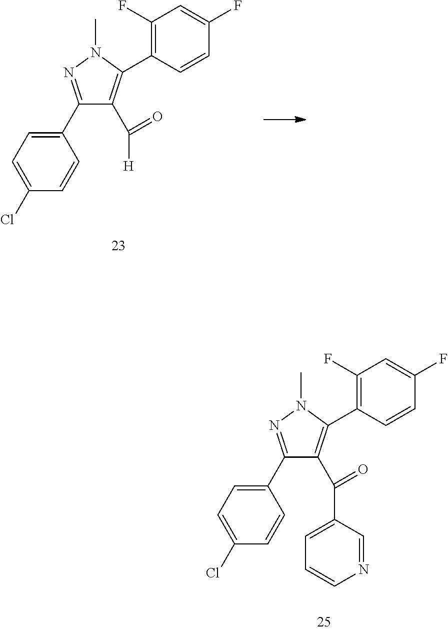

A solution of 3-bromopyridine (0.24 g) in 5 ml of THF (5 ml) is cooled to 0° C. under N₂ and to this is added drop-wise an isopropylmagnesium chloride lithium chloride complex solution (2.26 ml, 1 M in THF). After stirring for 2.5 hours at room temperature, a solution of 23 (0.41 g) in THF (5 ml) is added drop-wise. The reaction is allowed to reach room temperature and the stirring is continued for 3 hours. The reaction mixture is quenched with water and extracted twice with EtOAc (50 ml). The combined organic layers are washed with brine, dried and concentrated to afford a crude material that is purified by flash column chromatography (30% EtOAc in heptane). The desired compound 25 (0.23 g) is obtained as a white solid with melting point: 117-119° C.

### Example 3

### Preparation of [5-(4-Chloro-phenyl)-3-(2,4-difluoro-phenyl)-1-methyl-1H-pyrazol-4-yl]-pyridin-3-yl-methanol (26) (compound C84 see Table III)

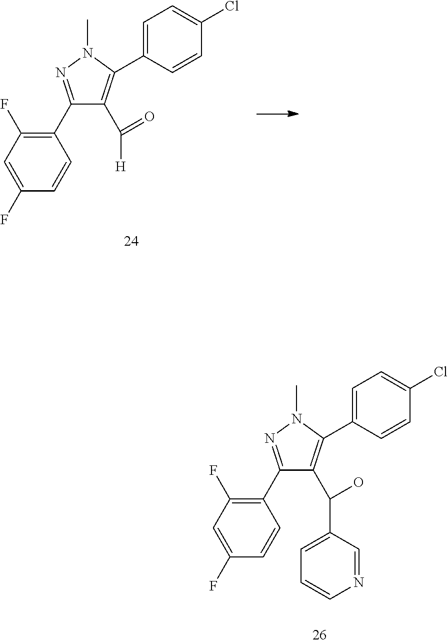

The same conditions reported for Step c (Example 2) are applied to 24 (0.43 g). The desired compound is isolated as white solid (0.40 g) with a melting point: 190-194° C.

### Biological Evaluation

### Example 4

### Biological Evaluation of Plant Growth Regulation Effects on Grape

5 weeks old grape seedlings cultivar (cv.) Gutedel were treated with the formulated test compound in a spray chamber. After an incubation period of 8 days at 22° C. and 80% relative humidity (r.h.) in a greenhouse, the plant growth regulating effects were assessed.

Compound A80 showed plant height decreased at 200 ppm.

### Example 5

### Biological Evaluation of Plant Growth Regulation Effects on Wheat

2 weeks old wheat plants cultivar (cv.). Riband were treated with the formulated test compound in a spray chamber. After an incubation period of 8 days at 22° C. and 80% relative humidity (r.h.) in a greenhouse, the plant growth regulating effects were assessed.

Compound B87 showed side shoot increased at 600 ppm.

1. Use of a compound of formula (I) 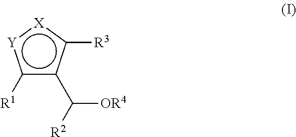
wherein
X is S, N or NR⁵ and Y is N or NR⁵, with the proviso that one, but not both, of X or Y is N;
R¹ and R³ are, independently, hydrogen, or optionally substituted alkyl, alkenyl, alkynyl, heterocyclyl, trialkylsilyl, arylalkyl, aryloxyalkyl, arylthioalkyl, aryl or heteroaryl;
R² is optionally substituted alkyl, alkenyl, alkynyl, heterocyclyl, arylalkyl, aryl or heteroaryl;
R⁴ is H or acyl;
R⁵ is hydrogen or optionally substituted alkyl, alkenyl, alkynyl, heterocyclyl, trialkylsilyl, arylalkyl, aryloxyalkyl, arylthioalkyl, aryl or heteroaryl;
or a salt or N-oxide thereof; as a plant growth regulator.

2. Use according to claim 1 wherein R¹ is selected from hydrogen; C₁-C₆-alkyl optionally substituted with phenyl; phenyl optionally substituted with halogen, C₁-C₆-alkyl, C₁-C₆-haloalkyl, C₁-C₆-alkoxy or C₁-C₆-haloalkoxy; and a 5- or 6-membered heteroaryl optionally substituted with halogen, C₁-C₆-alkyl, C₁-C₆-haloalkyl, C₁-C₆-alkoxy or C₁-C₆-haloalkoxy.

3. Use according to claim 2 wherein R¹ is selected from n-propyl, isopropyl, cyclohexyl, benzyl; phenyl optionally substituted with 1 or 2 groups independently selected from bromo, chloro, fluoro, methyl, methoxy, trifluoromethyl and trifluoromethoxy; and 2- or 3-pyridyl, 2- or 3-furyl, and 2- or 3-thienyl, each optionally substituted with 1 or 2 chloro.

4. Use according to claim 1 wherein R² is pyridyl or pyrimidinyl, each optionally substituted with halogen, C₁-C₆-alkyl, C₁-C₆-haloalkyl, C₁-C₆-alkoxy or C₁-C₆-haloalkoxy.

5. Use according to claim 4 wherein R² is selected from 2-pyridyl, 3-pyridyl, and 5-pyrimidinyl, each optionally substituted with methyl, chloro, fluoro or methoxy.

6. Use according to claim 1 wherein R³ is selected from hydrogen; C₁-C₆-alkyl optionally substituted with phenyl; phenyl optionally substituted with halogen, C₁-C₆-alkyl, C₁-C₆-haloalkyl, C₁-C₆-alkoxy or C₁-C₆-haloalkoxy; and a 5- or 6-membered heteroaryl optionally substituted with halogen, C₁-C₆-alkyl, C₁-C₆-haloalkyl, C₁-C₆-alkoxy or C₁-C₆-haloalkoxy.

7. Use according to claim 6 wherein R³ is selected from ethyl, isopropyl, isoamyl, cyclohexyl; phenyl optionally substituted with 1 or 2 groups independently selected from bromo, chloro, fluoro, methoxy and trifluoromethyl; and 2- or 3-furyl, and 2- or 3-thienyl, each optionally substituted with 1 or 2 chloro.

8. Use according to claim 1 wherein R⁴ is H.

9. Use according to claim 1 wherein R⁵ is selected from hydrogen; C₁-C₆-alkyl optionally substituted with phenyl; and phenyl optionally substituted with halogen, C₁-C₆-alkyl, C₁-C₆-haloalkyl, C₁-C₆-alkoxy or C₁-C₆-haloalkoxy.

10. Use according to claim 9 wherein R⁵ is methyl or benzyl.

11. Use according to claim 1 wherein X is S and Y is N.

12. Use according to claim 1 wherein X is NR⁵ and Y is N.

13. Use according to claim 1 wherein X is N and Y is NR⁵.

14. A compound, which is the (R)-enantiomer of the compound of formula (I) as defined in claim 1; and salts thereof.

15. A compound, which is the (S)-enantiomer of the compound of formula (I) as defined in claim 1; and salts thereof.

16. A method of regulating plant growth of crops of useful plants, which comprises applying to said plants, to one or more parts of said plants, or to the locus thereof or plant propagation material, a compound of formula (I) as defined in claim 1.

17. A method according to claim 16, which comprises one or more applications of one of more compounds of formula (I) alone or in conjunction with one or more customary plant protection formulating auxiliaries.

18. A method according to claim 17 wherein two or more applications are carried out in sequence, and wherein the two or more applications have the same or different concentration or combinations of compounds of formula (I) or both.

19. A method according to claim 16 wherein the useful crop plants are selected from the group consisting of cereals, rice, beets, leguminous plants, oil plants, cucumber plants, fibre plants, vegetables, plantation crops, ornamentals, vines, bushberries, caneberries, cranberries, peppermint, rhubarb, spearmint, sugar cane and turf grasses.

20. A method according to claim 16 wherein the plant growth regulating effect is an inhibition or a retardation of the plant growth.

21. An agricultural composition comprising a compound according to claim 14, or an agrochemically acceptable salt thereof, and an agrochemically acceptable diluent or carrier.

22. An agricultural composition comprising one or more compounds of formula (I) as defined in claim 1, and one or more customary plant protection auxiliaries.
